(19) United States
(12) Patent Application Publication
(10) Pub. No.: US 2026/0014170 A1
(43) Pub. Date: Jan. 15, 2026

(54) PRODRUGS FOR DELIVERY OF TESTOSTERONE TO THE CENTRAL NERVOUS SYSTEM

(71) Applicant: The Johns Hopkins University, Baltimore, MD (US)

(72) Inventors: Rana RAIS, West Friendship, MD (US); Barbara SLUSHER, Kingsville, MD (US); Mark Christopher MARKOWSKI, Baltimore, MD (US); Arindom PAL, Baltimore, MD (US)

(21) Appl. No.: 18/992,471

(22) PCT Filed: Jul. 28, 2023
(86) PCT No.: PCT/US2023/028998

§ 371 Date: Jan. 8, 2025

Related U.S. Application Data
(60) Provisional application No. 63/392,974, filed on Jul. 28, 2022.

(51) Int. Cl. A61K 31/58; A61K 31/568; A61K 45/06; C07J 1/00; C07J 41/00; C07J 43/00

(52) U.S. Cl. CPC A61K 31/58; A61K 31/568; A61K 45/06; C07J 1/0025; C07J 1/0029; C07J 41/0066; C07J 41/0088; C07J 43/003

(57) ABSTRACT

Prodrugs of testosterone and their use in alleviating, improving or reducing a risk of developing one or more conditions associated with androgen deprivation therapy (ADT), including orchiectomy or administration of a luteinizing hormone-releasing hormone agonist/antagonist, for the treatment of prostate cancer, including metastatic prostate cancer. In some aspects the prodrugs can be administered intranasally to target the central nervous system.

### BACKGROUND

Prostate cancer (PC) is the most common non-cutaneous malignancy in men with more than 190,000 new cases diagnosed in 2020 in the United States alone. Although disease confined to the prostate is highly curable, many men will be diagnosed with stage IV, or metastatic cancer. Metastatic PC is not curable, but treatable, with favorable long-term outcomes. The backbone of treatment for metastatic PC is lifelong, continuous androgen deprivation therapy (ADT). ADT is a form of castration in which circulating testosterone in the blood is reduced to nearly undetectable levels through surgery (i.e., orchiectomy) or medication (i.e., luteinizing hormone-releasing hormone agonist/antagonist).

Although effective at controlling the spread of PC, ADT can result in significant morbidity and worsen the quality of life of PC patients. Patients on ADT have a higher risk of bone fractures, cardiovascular events, cognitive deficits, sexual dysfunction, and severe fatigue. These symptoms can persist for the duration of therapy, which can be upwards of ten years or longer.

### SUMMARY

In some aspects, the presently disclosed subject matter provides a compound of formula (I):

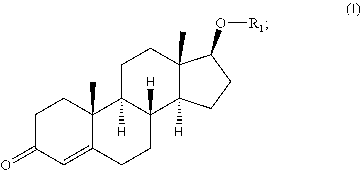

wherein:
R₁ is selected from —(CH₂)ₙ—O—C(═O)—O—R₂, —C(═O)—(CH₂)ₘ—NR₃R₄, —C(═O)—CH(R₅)—NR₆—C(═O)—CH(R₇)—R₈, —C(═O)—R₉, —C(═O)—(CH₂)ₘ—NR₁₀—C(═O)—(CH) p-NR₁₁R₁₂, —C(═O)—(CH₂)ₘ—NR₁₃—(C═O)—(CH)_{q}—NR₁₄—C(═O)—(CH₂)ₚ—NR₁₅R₁₆, —C(═O)—CH(R₁₇)—NR₁₈—C(═O)—(CH₂)ₚ—NR₁₉R₂₀, —C(═O)—(CH₂)ₘ—CHR₂₁—C(═O)—OH;
each n, m, and p is independently an integer selected from 0, 1, 2, 3, 4, 5, 6, 7, and 8; R₂, R₃, R₄, R₅, and R₈ are each independently straightchain or branched C₁-C₄ alkyl, or R₃ and R₄ together with the nitrogen atom to which they are bound form a substituted or unsubstituted 6-membered cycloheteroalkyl ring;
R₆, R₁₀, R₁₁, R₁₂, R₁₃, R₁₄, R₁₅, and R₁₆, R₁₈, and R₁₉, and R₂₀ are each independently H or C₁-C₄ alkyl;
R₇ is —NR₂₂R₂₃, wherein R₂₂ and R₂₃ are each independently H or C₁-C₄ alkyl;
R₉ is substituted or unsubstituted straightchain or branched C₁-C₄ alkyl;
R₁₇ is substituted or unsubstituted straightchain or branched C₁-C₈ alkyl;
R₂₁ is —NR₂₂R₂₃, wherein R₂₂ and R₂₃ are each independently H or —C(═O)—OR₂₄, wherein R₂₄ is C₁-C₄ alkyl; or
a pharmaceutically acceptable salt thereof.

In some aspects, the presently disclosed subject matter provides a pharmaceutical formulation comprising a compound of formula (I) and a pharmaceutically acceptable carrier. In certain aspects, the formulation is formulated for intranasal delivery.

In other aspects, the presently disclosed subject matter provides a method for treating a subject for prostate cancer, the method comprising administering to the subject a compound of formula (I), or a pharmaceutical formulation thereof, in combination with one or more additional therapies for prostate cancer. In certain aspects, the prostate cancer comprises stage IV prostate cancer or a metastatic prostate cancer. In particular aspects, the one or more additional therapies for prostate cancer comprises androgen deprivation therapy (ADT). In more particular aspects, the ADT is achieved through orchiectomy or administration of a luteinizing hormone-releasing hormone agonist/antagonist. In certain aspects, the method alleviates, improves, or reduces a risk of developing one or more conditions associated with ADT selected from a bone fracture, a cardiovascular event, a cognitive deficit, a sexual dysfunction, and severe fatigue.

In certain aspects, the compound of formula (I) is administered intranasally.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1 shows the in vitro stability of the presently disclosed testosterone prodrugs in brain homogenate;

FIG. 2A, FIG. 2B, and FIG. 2C demonstrate that the presently disclosed testosterone prodrugs release testosterone in brain homogenates; (FIG. 2A) Compound P1; (FIG. 2B) Compound P3; and (FIG. 2C) Compounds P1, P3, P5-P14 at 0, 30, and 60 min;

FIG. 3A shows testosterone release of prodrugs P1, P3-P14 in plasma and brain at 1 hour (h); and

FIG. 3B shows the Brain: Plasma ratio of released testosterone from prodrugs P1, P3-P14 at one hour (h).

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### A. Prodrugs of Testosterone

Without wishing to be bound to any one particular theory, it is thought that restoration of testosterone specifically in the CNS can attenuate several of the side effects of ADT while concomitantly maintaining castrate levels of testosterone in systemic circulation and avoid PC progression. To achieve this goal, the presently disclosed subject matter provides hydrophobic or lipophilic prodrugs of testosterone to be delivered to the CNS. One goal of the presently disclosed subject matter is to enhance brain penetration of testosterone while maintaining low systemic levels. The presently disclosed prodrugs exhibit two-to-three fold improvement (as much as 300%) in brain/plasma ratio compared to administration of testosterone at equimolar doses.

Accordingly, in some embodiments, the presently disclosed subject matter provides a compound of formula (I):

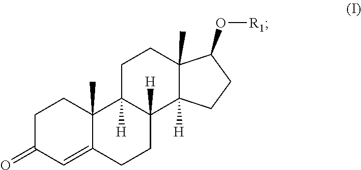

wherein:
R₁ is selected from —(CH₂)ₙ—O—C(═O)—O—R₂, —C(═O)—(CH₂)ₘ—NR₃R₄, —C(═O)—CH(R₅)-NR₆—C(═O)—CH(R₇)—R₈, —C(═O)—R₉, —C(═O)—(CH₂)ₘ—NR₁₀—C(═O)—(CH)ₚ—NR₁₁R₁₂, —C(═O)—(CH₂)ₘ—NR₁₃—(C═O)—(CH)_{q}—NR₁₄—C(═O)—(CH₂)ₚ—NR₁₅R₁₆, —C(═O)—CH(R₁₇)—NR₁₈—C(═O)—(CH₂)ₚ—NR₁₉R₂₀, —C(═O)—(CH₂)ₘ—CHR₂₁—C(—O)—OH;
each n, m, and p is independently an integer selected from 0, 1, 2, 3, 4, 5, 6, 7, and 8;
R₂, R₃, R₄, R₅, and R₈ are each independently straightchain or branched C₁-C₄ alkyl, or R₃ and R₄ together with the nitrogen atom to which they are bound form a substituted or unsubstituted 6-membered cycloheteroalkyl ring;
R₆, R₁₀, R₁₁, R₁₂, R₁₃, R₁₄, R₁₅, and R₁₆, R₁₈, and R₁₉, and R₂₀ are each independently H or C₁-C₄ alkyl;
R₇ is —NR₂₂R₂₃, wherein R₂₂ and R₂₃ are each independently H or C₁-C₄ alkyl;
R₉ is substituted or unsubstituted straightchain or branched C₁-C₄ alkyl;
R₁₇ is substituted or unsubstituted straightchain or branched C₁-C₈ alkyl;
R₂₁ is —NR₂₂R₂₃, wherein R₂₂ and R₂₃ are each independently H or —C(═O)—OR₂₄, wherein R₂₄ is C₁-C₄ alkyl; or
a pharmaceutically acceptable salt thereof.

In certain embodiments, R₁ is —(CH₂)ₙ—O—C(—O)—O—R₂, wherein n is 1 and R₂ is selected from methyl, ethyl, n-propyl, isopropyl, n-butyl, isobutyl, sec-butyl, and tert-butyl. In particular embodiments, R₂ is isopropyl.

In certain embodiments, R₁ is —C(═O)—(CH₂)ₘ—NR₃R₄, wherein m is 1 and R₃ and R₄ are each independently selected from methyl, ethyl, n-propyl, isopropyl, n-butyl, isobutyl, sec-butyl, and tert-butyl. In particular embodiments, R₃ and R₄ are each methyl. In other embodiments, R₃ and R₄ together with the nitrogen atom to which they are bound form a 4-methylpiperazinyl or morpholinyl ring.

In certain embodiments, R₁ is —C(═O)—CH(R₅)—NR₆—C(═O)—CH(R₇)—R₈, wherein R₆ is H, R₇ is —NH₂, and R₅ and R₅ are each independently selected from methyl, ethyl, n-propyl, isopropyl, n-butyl, isobutyl, sec-butyl, and tert-butyl. In particular embodiments, R₅ and R₈ are each isopropyl.

In some embodiments, R₁ is —C(═O)—R₉, wherein R₉ is C₁-C₄ alkyl substituted with halogen. In particular embodiments, R₉ is —CH(Cl)₂.

In some embodiments, R₁ is —C(═O)—(CH₂)ₘ—NR₁₀—C(═O)—(CH)ₚ—NR₁₁R₁₂, wherein m and p are each 1, R₁₀ is H and R₁₁ and R₁₂ are each C₁-C₄ alkyl. In particular embodiments, R₁₁ and R₁₂ are each independently H or methyl.

In some embodiments, R₁ is —C(═O)—(CH₂)ₘ—NR₁₃—(C═O)—(CH)_{q}—NR₁₄—C(—O)—(CH₂)ₚ—NR₁₅R₁₆, wherein m, p, and q are each 1, R₁₃ and R₁₄ are each H and R₁₅ and R₁₆ are each C₁-C₄ alkyl. In particular embodiments, R₁₅ and R₁₆ are each methyl.

In some embodiments, R₁ is —C(═O)—CH(R₁₇)—NR₁₈—C(═O)—(CH₂)ₚ—NR₁₉R₂₀, wherein p is 1, R₁₈ is H, R₁₉ and R₂₀ are each C₁-C₄ alkyl, and R₁₇ is selected from methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, and benzyl. In particular embodiments, R₁₉ and R₂₀ are each methyl. In particular embodiments, R₁₇ is isobutyl. In particular embodiments, R₁₇ is benzyl.

In some embodiments, R₁ is —C(═O)—(CH₂)ₘ—CHR₂₁—C(═O)—OH, wherein m is 2 and R₂₁ is —NH₂ or —NH(COOCH₃).

In particular embodiments, the compound of formula (I) is selected from:

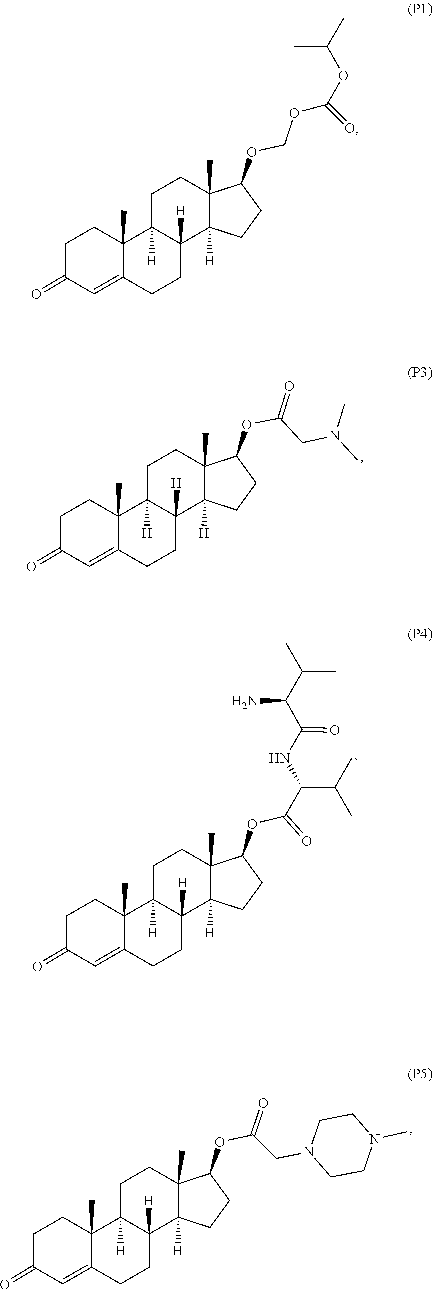

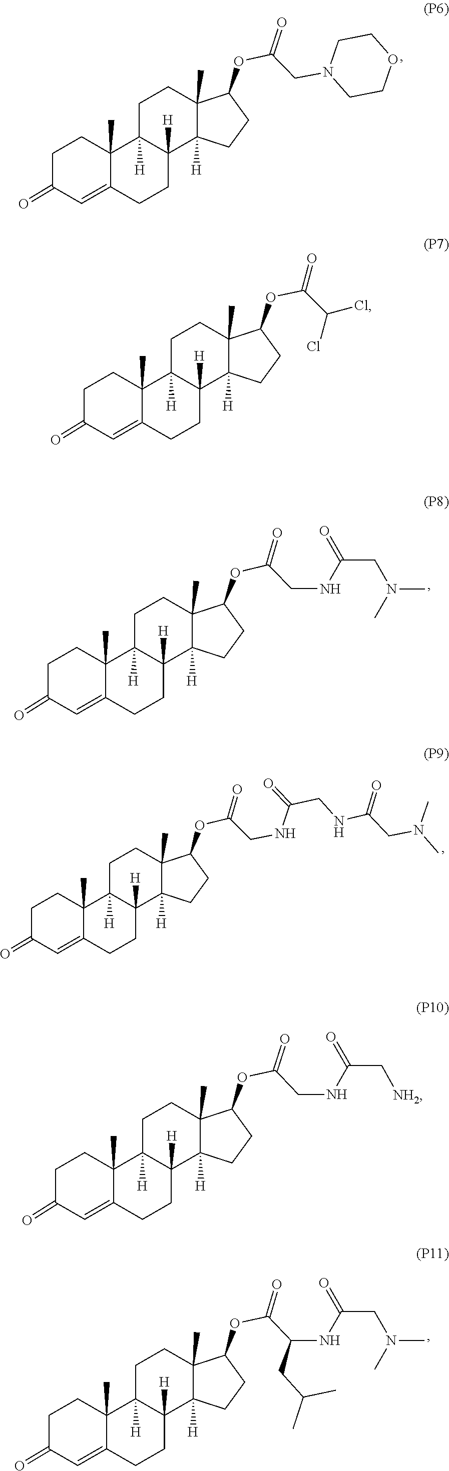

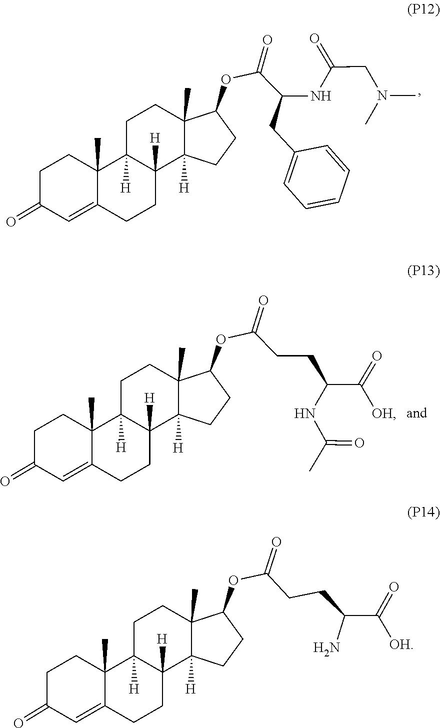

Representative testosterone prodrugs and their cLogP values are provided in Table 1.

TABLE 1
Testosterone Prodrugs and their MW and cLogP values

| Compound | Structure | MW | cLogP |
|---|---|---|---|
| Testosterone (1) | 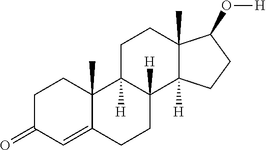 | 288.43 | 3.41 |
| P1 JHU5390 | 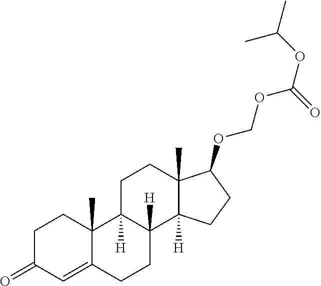 | 404.55 | 4.77 |
| P2 JHU5391 | 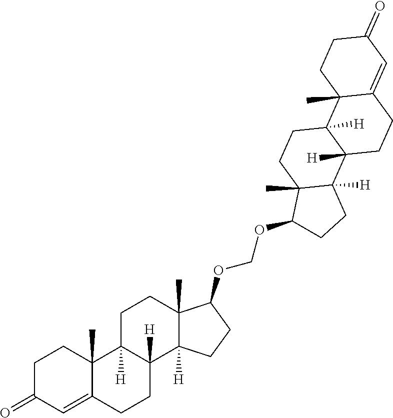 | 588.87 | 7.91 |
| P3 JHU5392 | 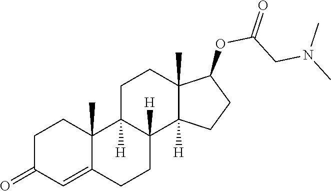 | 373.54 | 4.37 |
| P4 JHU5393 | 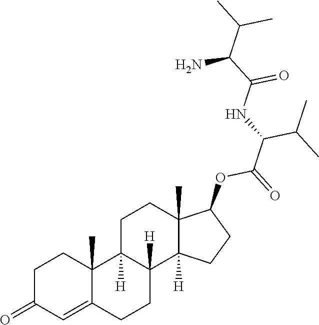 | 486.70 | 5.33 |
| P5 JHU5394 | 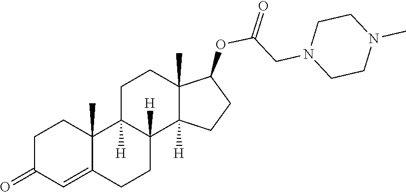 | 428.62 | 3.59 |
| P6 JHU5395 | 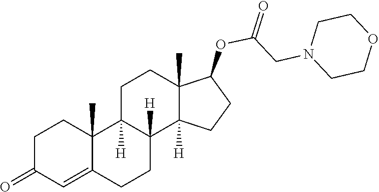 | 415.57 | 4.47 |
| P7 JHU5396 | 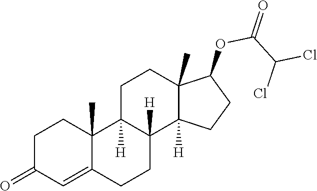 | 399.35 | 5.53 |
| P8 JHU5397 | 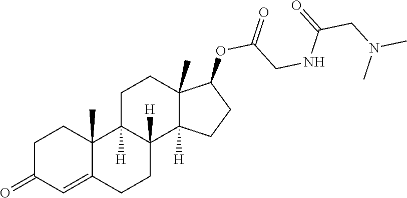 | 430.59 | 3.81 |
| P9 JHU5398 | 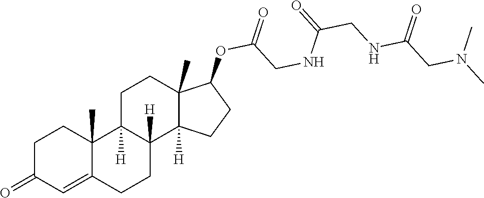 | 487.64 | 3.24 |
| P10 JHU5399 | 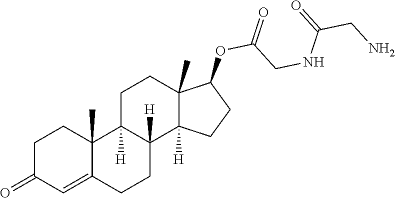 | 402.54 | 2.86 |
| P11 JHU5400 | 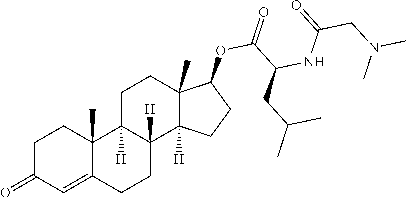 | 486.70 | 5.57 |
| P12 JHU5401 | 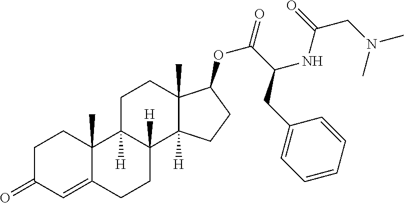 | 520.71 | 5.53 |
| P13 JHU5402 | 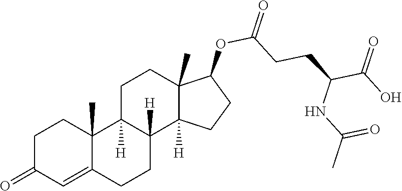 | 459.58 | 3.06 |
| P14 JHU5411 | 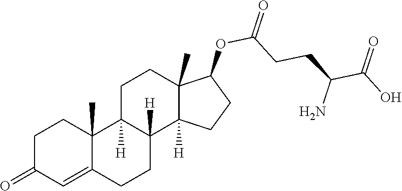 | 417.55 | 1.95 |

### B. Pharmaceutical Formulations of Compounds of Formula (I)

In some embodiments, the presently disclosed subject matter provides a pharmaceutical formulation comprising a compound of formula (I) and a pharmaceutically acceptable carrier.

Use of pharmaceutically acceptable inert carriers to formulate the compounds of formula (I) into dosages suitable for administration to a subject is within the scope of the disclosure. Depending on the specific conditions being treated, the presently disclosed compounds of formula (I) may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

In particular embodiments, however, the compounds of formula (I) are administered intranasally. For intranasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

In particular embodiments, the compound of formula (I) is administered intranasally in a form selected from the group consisting of a nasal spray, a nasal drop, a powder, a granule, a cachet, a tablet, an aerosol, a paste, a cream, a gel, an ointment, a salve, a foam, a paste, a lotion, a cream, an oil suspension, an emulsion, a solution, a patch, and a stick.

As used herein, the term administrating via an “intranasal route” refers to administering by way of the nasal structures. The presently disclosed testosterone prodrugs can be more effective at penetrating the central nervous system when administered intranasally. As used herein, the term “central nervous system” includes the brain and spinal cord.

Intranasal administration generally allows the active agent to bypass first pass metabolism, thereby enhancing the bioavailability of the active agent. Such delivery can offer several advantages over other modes of drug delivery, including, but not limited to, increasing the onset of action, lowering the required dosage, enhancing the efficacy, and improving the safety profile of the active agent. For example, tablet dosage forms enter the bloodstream through the gastrointestinal tract, which subjects the drug to degradation from stomach acid, bile, digestive enzymes, and other first pass metabolism effects. As a result, tablet formulations often require higher doses and generally have a delayed onset of action. Nasal administration of a drug also can facilitate compliance, especially for pediatric patients, geriatric patients, patients suffering from a neurodegenerative disease, or other patients for which swallowing is difficult, e.g., patients suffering from nausea, such as patients undergoing chemotherapy, or patients with a swallowing disorder.

Intranasal (“i.n.” or “IN”) delivery of an agent to a subject can facilitate delivery of the agent to the central nervous system. Such administration is non-invasive and offers several advantages including avoidance of hepatic first pass clearance, rapid onset of action, frequent self-administration and easy dose adjustments. Small molecules have an added advantage of being absorbed paracellularly through the nasal epithelium after which, these molecules can then directly enter the CNS through the olfactory or the trigeminal nerve associated pathway and can be directly transported to the brain upon intranasal administration.

For intranasal delivery, in addition to the active ingredients, pharmaceutical compositions may contain suitable pharmaceutically acceptable carriers comprising excipients and auxiliaries which facilitate processing of the active compounds into preparations which can be used pharmaceutically. The agents of the disclosure may be formulated by methods known to those of skill in the art, and may include, for example, but not limited to, examples of solubilizing, diluting, or dispersing substances, such as saline, preservatives, such as benzyl alcohol, absorption promoters, and fluorocarbons. Optimized formulations for intranasal delivery may include addition of permeability enhancers (mucoadhesives, nanoparticles, and the like) as well as combined use with an intranasal drug delivery device (for example, one that provides controlled particle dispersion with particles aerosolized to target the upper nasal cavity).

In particular, polymer-based nanoparticles, including chitosan, maltodextrin, polyethylene glycol (PEG), polylactic acid (PLA), polylactic-co-glycolic acid (PLGA), and PAMAM dendrimer; gels, including poloxamer; and lipid-based formulations, including glycerol monocaprate (Capmul™), mixtures of mono-, di-, and triglycerides and mono- and di-fatty esters of PEG (Labrafil™), palmitate, glycerol monostearate, and phospholipids can be used to administer the presently disclosed compounds of formula (I) intranasally.

The presently disclosed compounds of formula (I) also can be administered intranasally via mucoadhesive agents. Mucoadhesion is commonly defined as the adhesion between two materials, at least one of which is a mucosal surface. More particularly, mucoadhesion is the interaction between a mucin surface and a synthetic or natural polymer. Mucoadhesive dosage forms can be designed to enable prolonged retention at the site of application, providing a controlled rate of drug release for improved therapeutic outcome. Application of dosage forms to mucosal surfaces may be of benefit to drug molecules not amenable to the oral route, such as those that undergo acid degradation or extensive first-pass metabolism. Mucoadhesive materials suitable for use with nasal administration of the presently disclosed GCP-II inhibitors include, but are not limited to, soluble cellulose derivatives, such as hydroxypropyl methylcellulose (HPMC), hydroxypropyl cellulose (HPC), methylcellulose (MC), and carboxymethyl cellulose (CMC), and insoluble cellulose derivatives, such as ethylcellulose and microcrystalline cellulose (MCC), starch (e.g., Amioca®), polyacrylates, such as poly(acrylic acid) (e.g., Carbopol® 974P), functionalized mucoadhesive polymers, such as polycarbophil, hyaluronan, and amberlite resin, and chitosan (2-amino-2-deoxy-(1→4)-β-d-glucopyranan) formulations and derivatives thereof.

In some embodiments, the formulation also includes a permeability enhancer. As used herein, the term “permeability enhancer” refers to a substance that facilitates the delivery of a drug across mucosal tissue. The term encompasses chemical enhancers that, when applied to the mucosal tissue, render the tissue more permeable to the drug. Permeability enhancers include, but are not limited to, dimethyl sulfoxide (DMSO), hydrogen peroxide (H₂O₂), propylene glycol, oleic acid, cetyl alcohol, benzalkonium chloride, sodium lauryl sulphate, isopropyl myristate, Tween 80, dimethyl formamide, dimethyl acetamide, sodium lauroylsarcosinate, sorbitan monolaurate, methylsulfonylmethane, Azone, terpenes, phosphatidylcholine dependent phospholipase C, triacyl glycerol hydrolase, acid phosphatase, phospholipase A2, concentrated saline solutions (e.g., PBS and NaCl), polysorbate 80, polysorbate 20, sodium dodecanoate (C12), sodium caprate (CIO) and/or sodium palmitate (CI 6), tert-butyl cyclohexanol (TBCH), and alpha-terpinol.

In some embodiments, the intranasal administration is accomplished via a ViaNase™ device (Kurve Technology, Inc.). Such devices create a focused, turbulent flow that navigates the curved pathways of the nasal cavity, allowing access to the olfactory region.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, trifluoroacetic acid (TFA), and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

### C. Methods of Treatment

In other embodiments, the presently disclosed subject matter provides a method for treating a subject for prostate cancer, the method comprising administering to the subject a compound of formula (I), or a pharmaceutical formulation thereof, in combination with one or more additional therapies for prostate cancer. In certain embodiments, the prostate cancer comprises stage IV prostate cancer or a metastatic prostate cancer. In particular embodiments, the one or more additional therapies for prostate cancer comprises androgen deprivation therapy (ADT). In more particular embodiments, the ADT is achieved through orchiectomy or administration of a luteinizing hormone-releasing hormone agonist/antagonist. In certain embodiments, the method alleviates, improves, or reduces a risk of developing one or more conditions associated with ADT selected from a bone fracture, a cardiovascular event, a cognitive deficit, a sexual dysfunction, and severe fatigue.

In some embodiments, the compound of formula (I) is administered intranasally.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a compound described herein and at least one other therapeutic agent, such as a chemotherapeutic agent or ADT. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds described herein can be administered alone or in combination with adjuvants that enhance stability of the compounds, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound described herein and at least one additional therapeutic agent can be varied so long as the beneficial effects of the combination of these agents are achieved. Accordingly, the phrase “in combination with” refers to the administration of a compound described herein and at least one additional therapeutic agent either simultaneously, sequentially, or a combination thereof. Therefore, a subject administered a combination of a compound described herein and at least one additional therapeutic agent can receive a compound and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound described herein and at least one additional therapeutic agent are administered simultaneously, they can be administered to the subject as separate pharmaceutical compositions, each comprising either a compound or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( SI )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

### D. Definitions

Unless otherwise noted, the chemical definitions provided immediately herein below are intended to comply with IUPAC. Compendium of Chemical Terminology, 2nd ed. (the “Gold Book”). Compiled by A. D. McNaught and A. Wilkinson. Blackwell Scientific Publications, Oxford (1997).

The term “hydrocarbon” as used herein, refers to any chemical group comprising hydrogen and carbon. A hydrocarbon group may be substituted or unsubstituted. As would be known to one of ordinary skill in the art, all valencies must be satisfied in making any substitutions. The hydrocarbon may be unsaturated, saturated, branched, unbranched, cyclic, polycyclic, or heterocyclic.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocyclyl”, “cycloaliphatic”, or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In some embodiments, aliphatic groups contain 1-4 aliphatic carbon atoms. In some embodiments, aliphatic groups contain 1-3 aliphatic carbon atoms. In some embodiments, aliphatic groups contain 1-2 aliphatic carbon atoms. In some embodiments, “cycloaliphatic” (or “carbocyclyl” or “cycloalkyl”) refers to a monocyclic C3-C7 hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl, or (cycloalkyl)alkenyl.

The term “alkane” refers to acyclic branched or unbranched hydrocarbons having the general formula CₙH₂ₙ₊₂, and therefore consisting entirely of hydrogen atoms and saturated carbon atoms.

The term “alkyl” refers to a univalent group derived from an alkane by removal of a hydrogen atom from any carbon atom and having the chemical formula of —CₙH₂ₙ₊₁. The groups derived by removal of a hydrogen atom from a terminal carbon atom of unbranched alkanes form a subclass of normal alkyl(n-alkyl) groups H(CH₂)ₙ. The groups RCH₂, R₂CH (R≠H), and R₃C(R≠H) are primary, secondary and tertiary alkyl groups, respectively.

An alkyl can be a straightchain (i.e., unbranched) or branched acyclic hydrocarbon having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C1-20 inclusive, including 1, 2, 3, 4, 5, 6, 7, 8, 9, 10, 11, 12, 13, 14, 15, 16, 17, 18, 19, and 20 carbons. In other embodiments, the alkyl can be a C₁-C₄ alkyl, including 1, 2, 3, and 4 carbons. In yet other embodiments, the alkyl can be a C₁-C₆ alkyl, including 1, 2, 3, 4, 5, and 6 carbons. In even yet other embodiments, the alkyl can be a C₁-C₈ alkyl, including 1, 2, 3, 4, 5, 6, 7, and 8 carbons.

“Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers to straight-chain alkyls. In other embodiments, “alkyl” refers to branched alkyls. In certain other embodiments, “alkyl” refers to straight-chain and/or branched alkyls. “Branched” refers to an alkyl group in which a lower alkyl group, such as methyl, ethyl or propyl, is attached to a linear alkyl chain.

Representative alkyl groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, and dodecyl.

Alkyl groups can optionally be substituted (a “substituted alkyl”) with one or more substituents, which can be the same or different. Such substituent groups include, but are not limited to, alkyl, substituted alkyl, cycloalkyl, halogen, acyl, carboxyl, oxo, aryl, substituted aryl, alkoxyl, hydroxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms consisting of at least one carbon atom and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

The term “cycloalkane” refers to saturated monocyclic hydrocarbons (with or without side chains), e.g., cyclobutane. Unsaturated monocyclic hydrocarbons having one endocyclic double or one triple bond are called cycloalkenes and cycloalkynes, respectively. Those having more than one such multiple bond are cycloalkadienes, cycloalkatrienes, and the like. The inclusive terms for any cyclic hydrocarbons having any number of such multiple bonds are cyclic olefins or cyclic acetylenes.

The term “cycloalkyl” refer to a univalent group derived from a cycloalkane by removal of a hydrogen atom from a ring carbon atom. Cycloalkyls can be a mono-or multicyclic ring system of about 3 to about 10 carbon atoms, e.g., 3, 4, 5, 6, 7, 8, 9, or 10 carbon atoms. The cycloalkyl group also can be optionally substituted with a substituent group provided hereinabove for alkyl groups. Representative monocyclic cycloalkyl rings include cyclopropyl, cyclobutyl, cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl” as used herein, refers to a cycloalkyl group, which is attached to the parent molecular moiety through an alkylene moiety, also as defined above, e.g., a C₁₋₂₀ alkylene moiety. Examples of cycloalkylalkyl groups include cyclopropylmethyl and cyclopentylethyl.

The terms “cycloheteroalkyl” and “heterocycloalkyl” (or more generally “heterocyclic”) are used interchangeably and refer to an unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur(S), phosphorus (P), and silicon (Si), in which the nitrogen, sulfur, and phosphorus heteroatoms may be oxidized and the nitrogen heteroatom may be quaternized. The cycloheteroalkyl ring can be optionally fused to or otherwise attached to other cycloheteroalkyl rings and/or non-aromatic hydrocarbon rings. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

The terms “cycloalkylene” and “heterocycloalkylene” refer to the divalent derivatives of cycloalkyl and heterocycloalkyl, respectively.

As used herein the terms “bicycloalkyl” and “bicycloheteroalkyl” refer to two cycloalkyl or cycloheteroalkyl groups that are bound to one another. Non-limiting examples include bicyclohexane and bipiperidine.

An “unsaturated hydrocarbon” has one or more double bonds or triple bonds. As used herein, the term “alkene” refers to an acyclic branched or unbranched hydrocarbons having one carbon-carbon double bond and the general formula CₙH₂ₙ. Acyclic branched or unbranched hydrocarbons having more than one double bond are alkadienes, alkatrienes, and the like.

More particularly, the term “alkenyl” as used herein refers to a monovalent group derived from a C₂₋₂₀ inclusive straight or branched hydrocarbon moiety having at least one carbon-carbon double bond by the removal of a single hydrogen molecule. Alkenyl groups include, but are not limited to, ethenyl (i.e., vinyl), 2-propenyl, butenyl, 1-methyl-2-buten-1-yl, pentenyl, 2-isopentenyl, hexenyl, octenyl, allenyl, butadienyl, crotyl(but-2-en-1-yl), 2-(butadienyl), 2,4-pentadienyl, 3-(1,4-pentadienyl), and the like, including higher homologs and isomers.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkyne” as used herein refers to an acyclic branched or unbranched hydrocarbons having a carbon-carbon triple bond and the general formula CₙH₂ₙ₋₂, RC≡CR. Acyclic branched or unbranched hydrocarbons having more than one triple bond are known as alkadiynes, alkatriynes, and the like.

The term “alkynyl” as used herein refers to a monovalent group derived from a straight or branched C₂₋₂₀ hydrocarbon of a designed number of carbon atoms containing at least one carbon-carbon triple bond. Examples of alkynyl groups include, but are not limited to, ethynyl, 2-propynyl (propargyl), 1-propynyl, pentynyl, hexynyl, and heptynyl groups, and the like.

As used herein, the term “alkylene” refers to an alkanediyl group having the free valencies on adjacent carbon atoms, e.g. CH(CH₃)CH₂-propylene (systematically called propane-1,2-diyl). More particularly, the term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂CH₂); propylene ((CH₂)₃); cyclohexylene (C₆H₁₀—); —CH═CH—CH═CH—; —CH═CH—CH₂; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂) 2—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂-and-CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula-C(O)OR′-represents both-C(O)OR′-and-R′OC(O)—.

The term “arene” refers to a monocyclic and polycyclic aromatic hydrocarbon.

The term “aryl” refers to a group derived from arenes by removal of a hydrogen atom from a ring carbon atom. Groups similarly derived from heteroarenes are sometimes subsumed in this definition. An aryl group can include, for example, a single ring or multiple rings (such as from 2 to 3 rings), which are fused together or linked covalently.

The term “heteroaryl” refers to a group formed by removing one or more hydroxy groups from oxoacids that have the general structure RₖE(═O)ₗ(OH)ₘ(l≠0), and replacement analogues of such acyl groups. In organic chemistry an unspecified acyl group is commonly a carboxylic acyl group.

The term “heteroaryl” refers to the class of heterocyclyl groups derived from heteroarenes by removal of a hydrogen atom from any ring atom. A “heteroaryl” group can include from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy) propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Each of above terms defined hereinabove (e.g., “alkyl,” “heteroalkyl,” “cycloalkyl, and “heterocycloalkyl”, “alkenyl”, “alkynyl,” “aryl,” “heteroaryl,” as well as their divalent derivatives) are meant to include both substituted and unsubstituted forms of the indicated group. Optional substituents for each type of group are provided below.

As used herein, the term “acyl” refers to a group formed by removing one or more hydroxy groups from oxoacids that have the general structure RₖE(═O)ₗ(OH)ₘ(l≠0), and replacement analogues of such acyl groups. In organic chemistry an unspecified acyl group is commonly a carboxylic acyl group. For example, in some embodiments, the term acyl includes an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl) acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O—and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(—O)—group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(—O)—group. Exemplary aryloxycarbonyl groups include phenoxy- and naphthoxy-carbonyl.

“Aralkoxycarbonyl” refers to an aralkyl-O—C(═O)—group. An exemplary aralkoxycarbonyl group is benzyloxycarbonyl.

The term “acyloxyl” refers to an oxygen-centered radicals consisting of an acyl radical bonded to an oxygen atom, e.g., an acyl-O— group wherein acyl is as previously described.

The term “amine” refers to a compound formally derived from ammonia by replacing one, two or three hydrogen atoms by hydrocarbyl groups, and having the general structures RNH₂ (primary amines), R₂NH (secondary amines), R₃N (tertiary amines). In some embodiments, the term amino refers to the —NH₂ group. More generally, the amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms “acylamino” and “alkylamino” refer to specific N-substituted organic radicals with acyl and alkyl substituent groups, respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ—where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The terms alkylthioether and thioalkoxyl refer to a saturated (i.e., alkyl-S—) or unsaturated (i.e., alkenyl-S—and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to a compound containing the carbonyl group, —C(═O)—. The term is commonly used in the restricted sense of aldehydes (R—C(═O) H) and ketones, although it actually includes carboxylic acids and derivatives.

The term “carboxylic acid” refers to an oxoacids having the structure RC(═O) OH. The term is used as a suffix in systematic name formation to denote the C(═O) OH group including its carbon atom. In some embodiments, the term “carboxyl” refers to the —COOH group. Such groups also are referred to herein as a “carboxylic acid” moiety.

“Carbamoyl” refers to an amide group of the formula —C(═O) NH₂.

“Alkylcarbamoyl” refers to a R′RN—C(—O)—group wherein one of R and R′ is hydrogen and the other of R and R′ is alkyl and/or substituted alkyl as previously described.

“Dialkylcarbamoyl” refers to a R′RN—C(═O)—group wherein each of R and R′ is independently alkyl and/or substituted alkyl as previously described.

The term carbonyldioxyl, as used herein, refers to a carbonate group of the formula —O—C(═O)—OR.

The term “cyano” refers to the —C═N group.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁₋₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo compound” refers to a compounds containing an oxygen atom, ═O, doubly bonded to carbon or another element. The term thus embraces aldehydes, carboxylic acids, ketones, sulfonic acids, amides and esters. Oxo used as an adjective (and thus separated by a space) modifying another class of compound, as in oxo carboxylic acids, indicates the presence of an oxo substituent at any position. To indicate a double-bonded oxygen that is part of a ketonic structure, the term keto is sometimes used as a prefix, but such use has been abandoned by IUPAC for naming specific compounds. A traditional use of keto is for indicating oxidation of CHOH to C═O in a parent compound that contains OH groups, such as carbohydrates, e.g. 3-ketoglucose. In some embodiments, the term “oxo” as used herein means an oxygen atom that is double bonded to a carbon atom or to another element.

The term “nitro” refers to the —NO₂ group.

The term “thio” refers to replacement of an oxygen by a sulfur, e.g., PhC(═S)NH₂, thiobenzamide.

The term “thiol” refers to a compounds having the structure RSH(R≠H), e.g., MeCH₂SH ethanethiol. A thiol also is known by the term “mercaptan.”

The term “thiohydroxyl” or “thiol,” as used herein, refers to a group of the formula —SH.

The term “sulfate” refers to the —SO₄ group.

The term “sulfide” refers to a compound having the structure RSR(R≠H) and also are referred to as “thioethers.”

The term “sulfone” refers to a compound having the structure, RS(═O)₂R(R≠H), e.g., C₂H₅S(═O)₂CH₃ ethyl methyl sulfone.

The term “sulfoxide” refers to a compound having the structure R₂S═O(R≠H), e.g., Ph₂S═O diphenyl sulfoxide.

The term “ureido” refers to a urea group of the formula —NH—CO—NH₂.

One of ordinary skill in the art would recognize that a structure represented generally by, for example, the formula:

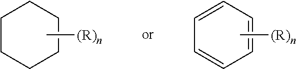

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

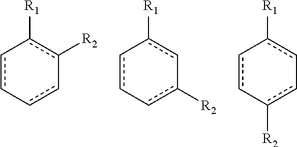

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or(S)-or, as D-or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

As used herein, the term “congener” refers to one of two or more substances related to each other by origin, structure, or function.

The term “enantiomer” refers to one of a pair of molecular entities which are mirror images of each other and non-superposable.

The term “stereoisomer” refers to an isomer that possess identical constitution, but which differ in the arrangement of their atoms in space.

The term “racemate” refers to an equimolar mixture of a pair of enantiomers. It does not exhibit optical activity. The chemical name or formula of a racemate is distinguished from those of the enantiomers by the prefix (±)- or rac-(or racem-) or by the symbols RS and SR.

The term “diastereoisomerism” refers to stereoisomerism other than enantiomerism. Diastereoisomers (or diastereomers) are stereoisomers not related as mirror images. Diastereoisomers are characterized by differences in physical properties, and by some differences in chemical behavior towards achiral as well as chiral reagents.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Synthetic Methods for Prodrugs P1-P14

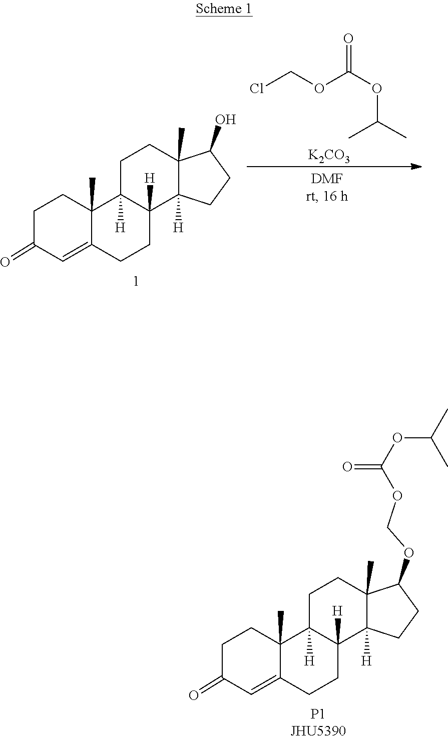

(((8R,9S,10R,13S,14S,17S)-10,13-Dimethyl-3-oxo-2,3,6,7,8,9,10,11,12,13,14,15,16,17-tetradecahydro-1H-cyclopenta[α]phenanthren-17-yl)oxy)methyl isopropyl carbonate (P1,

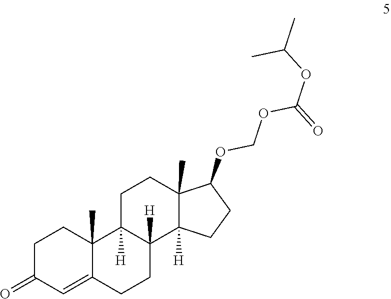

To a stirred solution of testosterone 1 (1.00 g, 3.40 mmol, 1.0 equiv.) in DMF (20 mL) was added potassium carbonate (2.25 g, 6.90 mmol, 2.0 equiv.) followed by chloromethyl isopropyl carbonate (1.05 g, 6.90 mmol, 2.0 equiv.). The reaction mixture was stirred at rt for 16 hand then was quenched with water and the crude product was extracted with ethyl acetate (2×150 mL). The combined organic layer was dried over anhydrous sodium sulphate, filtered and concentrated under reduced pressure. The residue was purified by column chromatography over silica gel (60-120 mesh) using 10% ethyl acetate in hexane as eluent to afford compound P1 (JHU5390) as a colorless solid (850 mg) in 65% yield.

¹H-NMR (400 MHZ, do-DMSO): 0.72 (s, 3H), 0.89-0.97 (m, 3H), 1.08-1.11 (m, 1H), 1.15 (s, 3H), 1.22-1.26 (m, 6H), 1.29-1.31 (m, 1H), 1.32-1.49 (m, 1H), 1.51-1.59 (m, 5H), 1.76-1.79 (m, 2H), 1.96-1.98 (m, 2H), 2.18-2.25 (m, 1H), 2.36-2.37 (m, 1H), 2.39-2.41 (m, 2H), 3.33-3.58 (m, 1H), 4.77-4.80 (m, 1H), 5.19-5.25 (m, 2H), 5.63 (s, 1H). ESI MS: 405.4 ([M+H]⁺).

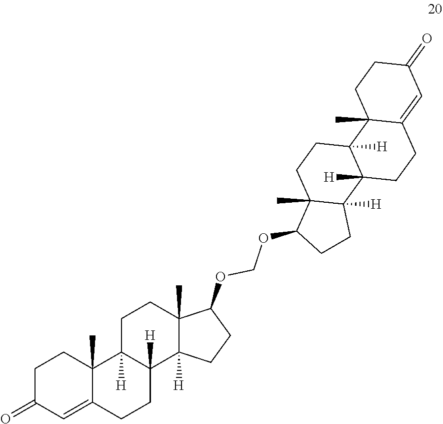

(8S,9R,10S,13R,14R,17R)-17-((((8R,9S,10R,13S,14S,17S)-10,13-Dimethyl-3-oxo-2,3,6,7,8,9,10,11,12,13,14,15,16,17-tetradecahydro-1H-cyclopenta[α]phenanthren-17-yl)oxy) methoxy)-10,13-dimethyl-1,2,6,7,8,9,10,11,12,13,14,15,16,17-tetradecahydro-3H-cyclopenta[α]phenanthren-3-one (P2, JHU5391)

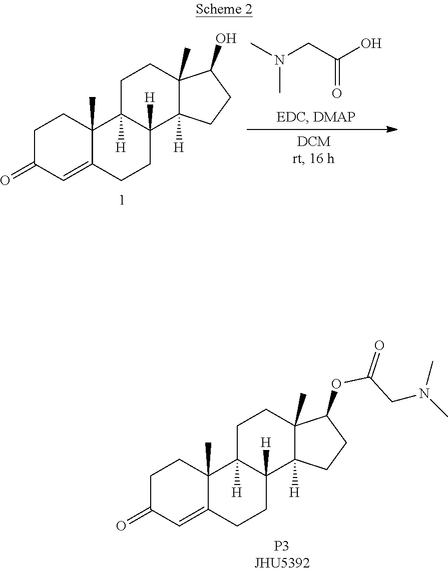

(8R,9S,10R,13S,14S,17S)-10,13-Dimethyl-3-oxo-2,3,6,7,8,9,10,11,12,13,14,15,16,17-tetradecahydro-1H-cyclopenta[α]phenanthren-17-yl dimethylglycinate (P3, JHU5392)

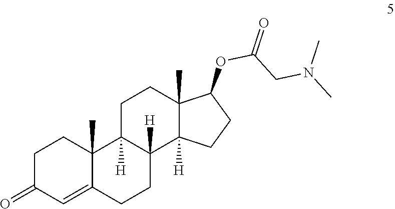

To a stirred solution of dimethylglycine (711 mg, 6.90 mmol, 1.7 equiv.) in DCM (10 mL) were added DMAP (562 mg, 4.00 mol, 1.0 equiv.) and EDC·HCl (2.21 g, 11.0 mmol, 2.8 equiv.), followed by testosterone 1 (1.00 g, 4.00 mol, 1.0 equiv.). The reaction mixture was stirred at rt for 16 h and then the mixture was concentrated under reduced pressure. The residue was purified by column chromatography over silica gel (60-120 mesh) using 48% ethyl acetate in hexane as eluent to afford compound P3 (JHU5392) as a colorless solid (1.00 g) in 77% yield.

¹H-NMR (400 MHZ, do-DMSO): 0.81 (s, 3H), 0.92-0.95 (m, 2H), 1.09 (m, 1H), 1.16-1.18 (m, 4H), 1.31-1.37 (m, 2H), 1.50-1.63 (m, 6H), 1.81 (m, 1H), 1.95 (m, 1H), 2.14-2.27 (m, 2H), 2.37-2.40 (m, 7H), 2.40-2.44 (m, 2H), 3.16 (s, 2H), 4.56-4.60 (m, 1H), 5.64 (s, 1H). ESI MS: 374.4 ([M+H]⁺).

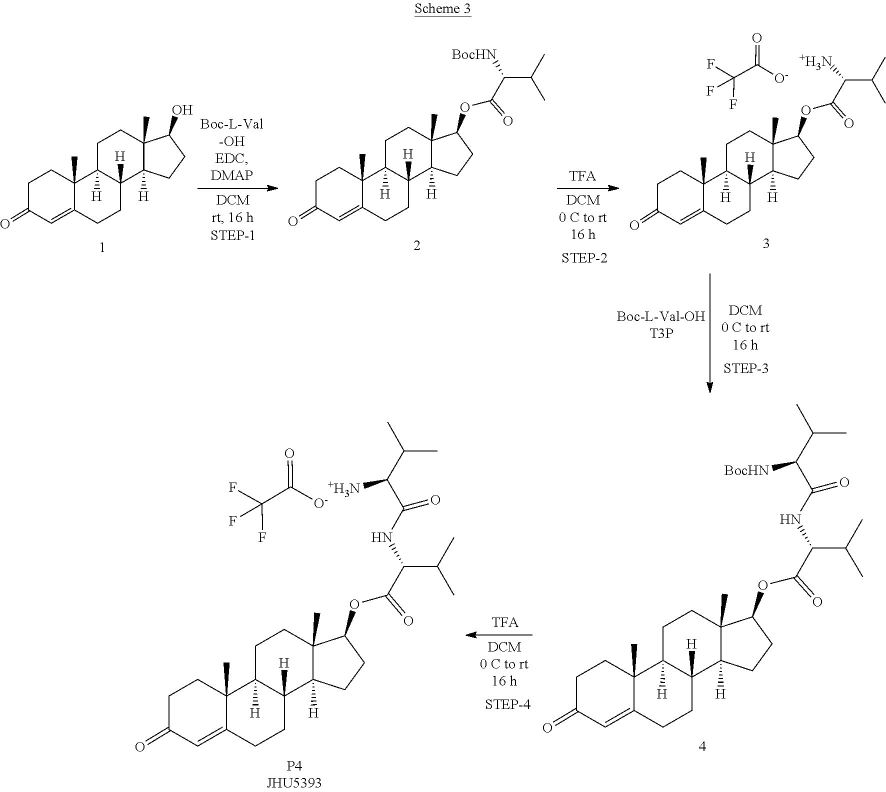

### Step 1:

Boc-L-Val-OH (1.00 g, 4.00 mmol, 1.0 equiv.) was dissolved in anhydrous DCM (10 mL) and DMAP (560 mg, 4.00 mmol, 1.0 equiv.) and EDC*HCl (2.21 g, 11.0 mmol, 2.8 equiv.), followed by testosterone 1 (1.99 g, 6.9 mmol, 1.7 equiv.) were added. The reaction mixture was stirred at rt. for 16 h and then was concentrated under reduced pressure. The residue was purified by column chromatography over silica gel (60-120 mesh) using 10% ethyl acetate in hexane as eluent to afford compound 2 as a colorless amorphous solid (2.0 g) in 89% yield.

### Step 2:

To an ice cooled solution of compound 2 (2.00 g, 4.00 mmol, 1.0 equiv.) in DCM (15 ml) was added TFA (2 mL), the reaction mixture was slowly warmed to rt and stirred for 16 h. Volatiles were removed under reduced pressure and the residue was quenched with 10% solution of sodium bicarbonate and the product 3 was extracted with DCM (2×150 mL). The combined organic layer was dried over anhydrous sodium sulphate, filtered and concentrated under reduced pressure to afford compound 3 as an off-white amorphous solid (1.80 g) as a TFA salt and which was taken as such for next step without further purification.

### Step 3:

To an ice cooled solution of crude compound 3 (1.80 g, 4.00 mmol, 1 equiv.) in THF (20 mL) was added Boc-L-Val-OH (1.00 g, 4.00 mmol, 1 equiv.) and TEA (1.61 g, 11.0 mmol, 2.8 equiv.), followed by T3P (50% in ethyl acetate) (6 ml, 9.00 mmol, 2.3 equiv.). The reaction mixture was stirred at rt for 16 hand then was quenched with water (30 mL) and the product was extracted with ethyl acetate (2×150 mL). The combined organic layer was dried over anhydrous sodium sulphate, filtered and concentrated under reduced pressure to afford crude product which was purified by column chromatography over silica gel (60-120 mesh) using 15% ethyl acetate in hexane as eluent to afford compound 4 as an off-white amorphous solid (1.80 g) in 66% yield.

### Step 4:

(8R,9S,10R,13S,14S,17S)-10,13-Dimethyl-3-oxo-2,3,6,7,8,9,10,11,12,13,14,15,16,17-tetradecahydro-1H-cyclopenta[α]phenanthren-17-yl L-valyl-D-valinate (P4, JHU5393)

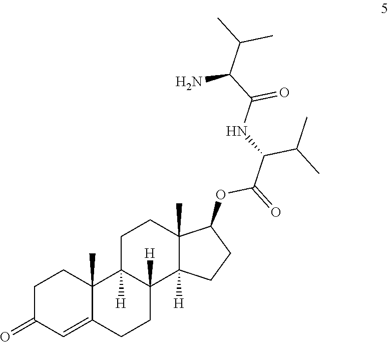

To an ice cooled solution of compound 4 (1.80 g, 3.00 mmol) in DCM (10 mL) was added TFA (2 mL). The reaction mixture was slowly warmed to rt and stirred for 16 h. Volatiles were removed under reduced pressure and the residue was purified by preparative HPLC to afford compound P4 (JHU5393) as a colorless solid (900 mg) in 60% yield as a TFA salt.

¹H-NMR (400 MHZ, do-DMSO): 0.84 (s, 3H), 0.91-0.93 (m, 10H), 0.96-0.98 (m, 4H), 1.03-1.15 (m, 1H), 1.16 (s, 3H), 1.38 (m, 2H), 1.50-1.62 (m, 5H), 1.62-1.64 (m, 1H), 1.70-1.73 (m, 1H), 1.96-2.06 (m, 1H), 2.11-2.18 (m, 4H), 2.37-2.38 (m, 1H), 2.41-2.50 (m, 2H), 3.76 (brs, 1H), 4.27-4.30 (m, 1H), 4.56 (t, J=8.0 Hz, 1H), 5.65 (s, 1H), 8.07 (s, 3H), 8.70 (d, J=8.4 Hz, 1H). ESI MS: 487.4 ([M+H]⁺).

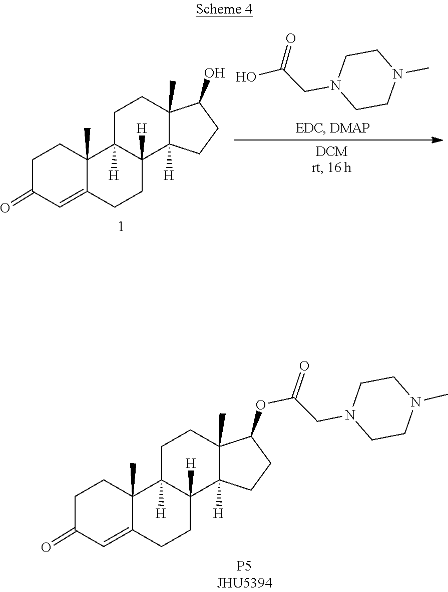

(8R,9S,10R,13S,14S,17S)-10,13-Dimethyl-3-oxo-2,3,6,7,8,9,10,11,12,13,14,15,16,17-tetradecahydro-1H-cyclopenta[α]phenanthren-17-yl 2-(4-methylpiperazin-1-yl)acetate (P5,

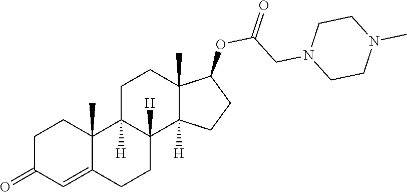

To a solution of testosterone 1 (500 mg, 1.73 mmol, 1.0 equiv.) and 2-(4-methylpiperazin-1-yl) acetic acid (549 mg, 3.47 mmol, 2.0 equiv.) in DCM (10 mL) was added EDC (997 mg, 5.20 mmol, 3.0 equiv.) and DMAP (63.5 mg, 0.520 mmol, 0.3 equiv.). The mixture was stirred at 25° C. for 16 h. The mixture was concentrated under reduced pressure. The residue was purified by prep-HPLC (column: Phenomenex Luna C18 200×40 mm×10 um; mobile phase: [water (HCl)−MeCN]; B %: 15%-45%, 15 min). Compound P5 (JHU5394) was obtained as a colorless solid (300 mg) in 40% yield.

¹H-NMR (400 MHZ, d₆-DMSO): 0.81 (3H, s), 0.87-1.01 (2H, m), 1.04-1.21 (5H, m), 1.27-1.40 (2H, m), 1.47-1.66 (5H, m), 1.72 (1H, d, J=12.4 Hz), 1.75-1.83 (1H, m), 1.92-2.00 (1H, m), 2.05-2.19 (2H, m), 2.25 (1H, d, J=14.0 Hz), 2.34-2.45 (2H, m), 2.34-2.45 (2H, m), 2.74 (3H, s), 2.94 (2H, brs), 3.15 (4H, brs), 3.42 (3H, d, J=10.8 Hz), 4.60 (1H, t, J=8.4 Hz), 5.63 (1H, s), 10.65-11.31 (1H, m). ESI MS: 429.3 ([M+H]⁺).

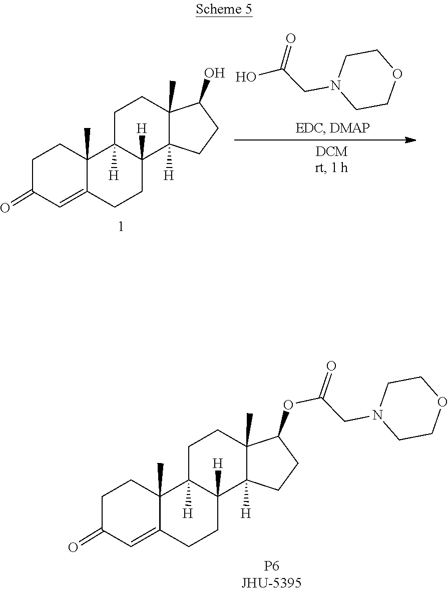

(8R,9S,10R,13S,14S,17S)-10,13-Dimethyl-3-oxo-2,3,6,7,8,9,10,11,12,13,14,15,16,17-tetradecahydro-1H-cyclopenta[α]phenanthren-17-yl 2-morpholinoacetate (P6, JHU5395)

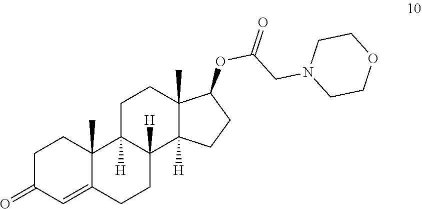

To a mixture of testosterone 1 (500 mg, 1.73 mmol, 1.0 equiv.), 2-morpholinoacetic acid (503 mg, 3.47 mmol, 2.0 equiv.) and EDC (997 mg, 5.20 mmol, 3.0 equiv.) in DCM (10 mL) was added DMAP (63.5 mg, 0.520 mmol, 0.3 equiv.) at 25° C. The mixture was stirred at 25° C. for 1 h. The reaction mixture was diluted with water (20 mL) and extracted with DCM (3×10 mL). The combined organic layers were washed with brine (3×20 mL), dried over anhydrous Na₂SO₄, filtered and concentrated under reduced pressure to give a residue. The residue was purified by flash silica gel chromatography (ISCO®; 20 g SepaFlash® Silica Flash Column, Eluent of 0˜25% EtOAc/Petroleum ether gradient @ 60 mL/min). Compound P6 (JHU5395) was obtained as a colorless solid (520 mg) in 66% yield.

¹H-NMR (400 MHZ, do-DMSO): 0.80 (3H, s), 0.86-1.01 (2H, m), 1.03-1.11 (1H, m), 1.15 (3H, s), 1.16-1.21 (1H, m), 1.29-1.39 (2H, m), 1.45-1.55 (2H, m), 1.56-1.71 (4H, m), 1.74-1.83 (1H, m), 1.92-2.00 (1H, m), 2.02-2.19 (2H, m), 2.21-2.29 (1H, m), 2.34-2.45 (2H, m), 2.46-2.49 (4H, m), 3.16-3.27 (2H, m), 3.52-3.62 (4H, m), 4.57 (1H, t, J=8.4 Hz), 5.63 (1H, s). ESI MS: 416.3 ([M+H]⁺).

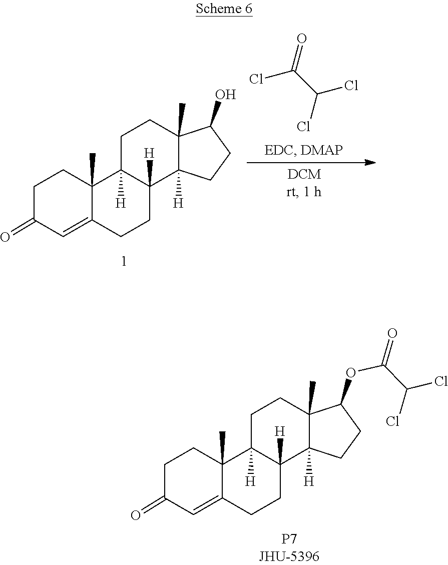

(8R,9S,10R,13S,14S,17S)-10,13-Dimethyl-3-oxo-2,3,6,7,8,9,10,11,12,13,14,15,16,17-tetradecahydro-1H-cyclopenta[α]phenanthren-17-yl 2,2-dichloroacetate (P7, JHU5396)

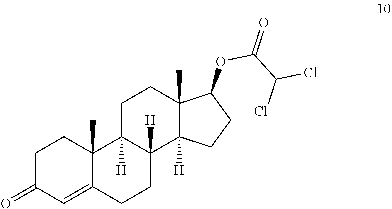

To a mixture of testosterone 1 (500 mg, 1.73 mmol, 1.0 equiv.), 2,2-dichloroacetyl chloride (511 mg, 334 μL, 3.47 mmol, 2.0 equiv.) and EDC (997 mg, 5.20 mmol, 3.0 equiv.) in DCM (10 mL) was added DMAP (63.5 mg, 0.520 mmol, 0.3 equiv). The mixture was stirred at 25° C. for 1 h. The reaction mixture was concentrated under reduced pressure. The residue was purified by prep-HPLC (column: Welch Ultimate XB-Diol 250×50×10 um; mobile phase: [Hexane-EtOH]; B %: 5%-25%, 40 min). Compound P7 (JHU5396) was obtained as a colorless solid (300 mg) in 43% yield

¹H-NMR (400 MHZ, do-DMSO): 0.84 (3H, s), 0.88-0.96 (2H, m), 1.15 (3H, s), 1.20-1.28 (2H, m), 1.30-1.45 (3H, m), 1.53-1.67 (5H, m), 1.79 (1H, d, J=12.4 Hz), 1.91-2.00 (1H, m), 2.10-2.19 (2H, m), 2.25 (1H, d, J=14.0 Hz), 2.36-2.41 (2H, m), 4.70 (1H, t, J=8.4 Hz), 5.51-5.77 (1H, m), 6.89 (1H, s). ESI MS: 399.1 ([M+H]⁺).

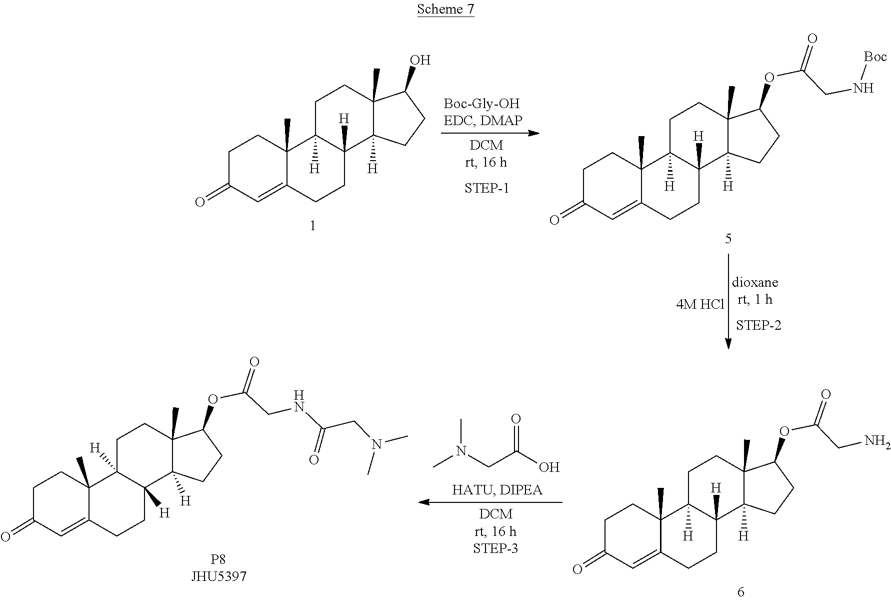

### Step 1:

To a solution of 2-(tert-butoxycarbonylamino) acetic acid (2.43 g, 13.9 mmol, 2.0 equiv.) in DCM (30 mL) was added EDCI (2.79 g, 14.6 mmol, 2.1 equiv.) and DMAP (84.7 mg, 0.693 mmol, 0.1 equiv) and finally testosterone 1 (2.00 g, 6.93 mmol, 1.0 equiv.) was added. The resulting mixture was stirred at 25° C. for 16 h. The mixture was concentrated under reduced pressure. The residue was purified by flash silica gel chromatography (ISCO®; 24 g SepaFlash® Silica Flash Column, eluent of 0˜10% MeOH: DCM gradient @ 60 mL/min). Compound 5 was obtained as an yellow oil (3.0 g) in 97% yield. ESI MS: 446.2 ([M+H]⁺).

### Step 2:

Compound 5 (3.00 g, 6.73 mmol, 1.0 equiv.) was dissolved in HCl/dioxane (4 M, 10 mL, 5.94 equiv.) and the mixture was stirred at 25° C. for 1 h. The volatiles were removed under reduced pressure. Compound 6 was obtained as a colorless solid (2.0 g) in 78% yield as a hydrochloride salt and the crude product was used to the following step without purification. ESI MS: 346.1 ([M+H]⁺).

### Step 3:

(8R,9S,10R,13S,14S,17S)-10,13-Dimethyl-3-oxo-2,3,6,7,8,9,10,11,12,13,14,15,16,17-tetradecahydro-1H-cyclopenta[α]phenanthren-17-yl dimethylglycylglycinate (P8, JHU5397)

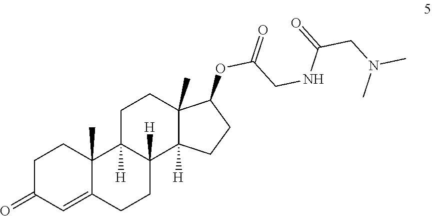

To a solution of compound 6 (700 mg, 1.83 mmol, 1.0 equiv), 2-(dimethylamino) acetic acid (378 mg, 3.67 mmol, 2.0 equiv) in DCM (15 mL) was added HATU (836 mg, 2.20 mmol, 1.2 equiv.) and DIPEA (711 mg, 958 μL, 5.50 mmol, 3.0 equiv). The mixture was stirred at 25° C. for 16 h. The mixture was concentrated under reduced pressure. The residue was purified by prep-HPLC (column: Welch Xtimate C18 150×25 mm×5 um; mobile phase: [water (HCl)-MeCN]; B %: 14%-44%, 9 min). Compound P8 (JHU5397) was obtained as a colorless solid (205 mg) in 24% yield.

¹H-NMR (400 MHZ, d₄-MeOD): 0.90 (s, 3H), 0.97-1.03 (m, 1H), 1.06-1.20 (m, 2H), 1.21-1.28 (m, 4H), 1.35-1.44 (m, 1H), 1.45-1.52 (m, 1H), 1.56-1.65 (m, 2H), 1.66-1.75 (m, 3H), 1.79 (brs, 1H), 1.88-1.94 (m, 1H), 2.05-2.11 (m, 1H), 2.14-2.22 (m, 1H), 2.26-2.36 (m, 2H), 2.44-2.53 (m, 2H), 2.95 (s, 6H), 4.00 (s, 2H), 4.03 (d, J=1.2 Hz, 2H), 4.69 (dd, J=8.0, 9.2 Hz, 1H), 5.72 (s, 1H). ESI MS: 431.4 ([M+Na]⁺).

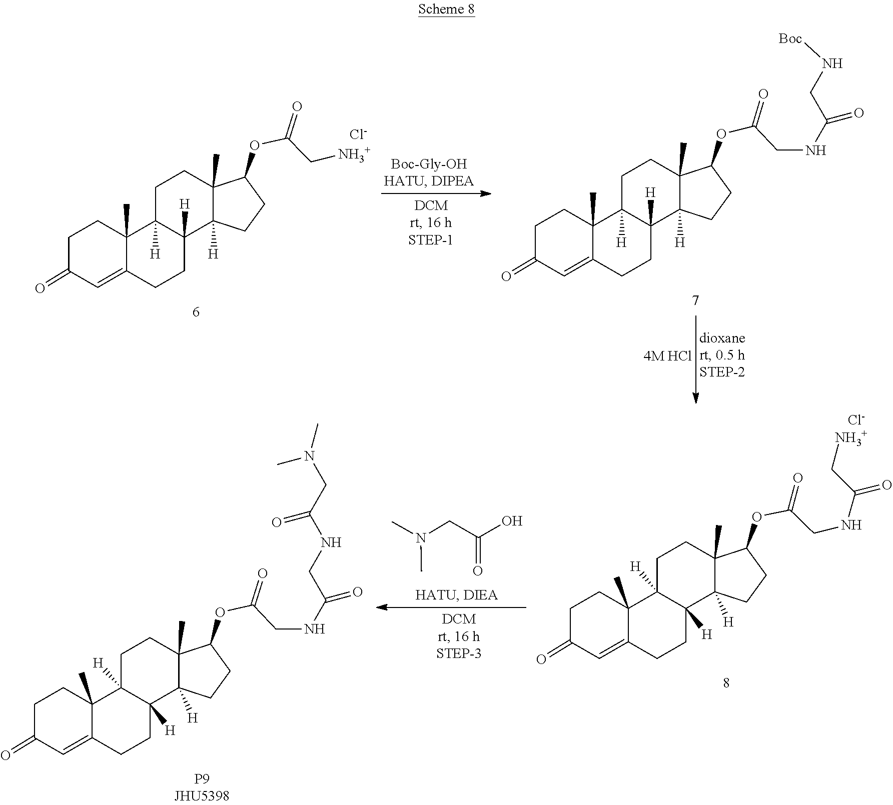

### Step 1:

To a solution of compound 6 (1.30 g, 3.40 mmol, 1.0 equiv.) and 2-(tert-butoxycarbonylamino) acetic acid (1.19 g, 6.81 mmol, 2.0 equiv) in DCM (15 mL) were added HATU (1.55 g, 4.08 mmol, 1.2 equiv) and DIPEA (1.32 g, 1.78 mL, 10.2 mmol, 3.0 equiv). The mixture was stirred at 25° C. for 16 h. The mixture was concentrated under reduced pressure. The residue was purified by flash silica gel chromatography (ISCO®; 24 g SepaFlash® Silica Flash Column, Eluent of 0˜100% EtOAc/Petroleum ether gradient @ 70 mL/min). Compound 7 was obtained as a yellow oil (1.50 g) in 88% yield. ESI MS: 447.2 ([M+H]⁺).

### Step 2:

Compound 7 (1.50 g, 2.78 mmol, 1.0 equiv.) was dissolved in 4M HCl in dioxane (10 mL, 14.4 equiv.) and the mixture was stirred at 25° C. for 0.5 h. The mixture was concentrated under reduced pressure. Compound 8 was obtained as a yellow solid (1.00 g) in 89% yield as a salt of hydrochloric acid and the crude product was used into the next step without further purification. ESI MS: 403.2 ([M+H]⁺).

### Step 3:

(8R,9S,10R,13S,14S,17S)-10,13-Dimethyl-3-oxo-2,3,6,7,8,9,10,11,12,13,14,15,16,17-tetradecahydro-1H-cyclopenta[α]phenanthren-17-yl dimethylglycylglycylglycinate (P9,

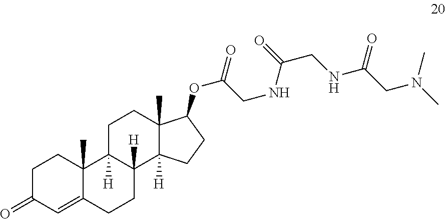

To a solution of compound 8 (1.00 g, 2.28 mmol, 1.0 equiv.) and 2-(dimethylamino) acetic acid (470 mg, 4.56 mmol, 2.0 equiv.) in DCM (20 mL) were added HATU (1.04 g, 2.73 mmol, 1.2 equiv.) and DIPEA (736 mg, 992 μL, 5.69 mmol, 2.5 equiv.). The mixture was stirred at 25° C. for 16 h. The mixture was concentrated under reduced pressure. The residue was purified by prep-HPLC (column: Phenomenex Luna C18 200×40 mm×10 um; mobile phase: [water (HCl)-MeCN]; B %: 15%-45%, 15 min) and then by prep-HPLC (column: Waters xbridge 150×25 mm 10 um; mobile phase: [water (NH₄HCO₃)-MeCN]; B %: 27%-57%, 8 min). Compound P9 (JHU5398) was obtained as a colorless solid (214 mg) in 19% yield.

¹H-NMR (400 MHZ, d₄-MeOD): 0.89 (3H, s), 0.96-1.21 (4H, m), 1.24 (3H, s), 1.34-1.49 (2H, m), 1.57-1.65 (2H, m), 1.66-1.75 (3H, m), 1.82 (1H, td, J=3.3, 12.6 Hz), 1.87-1.95 (1H, m), 2.05-2.12 (1H, m), 2.13-2.21 (1H, m), 2.28-2.40 (8H, m), 2.44-2.53 (2H, m), 3.05 (2H, s), 3.95 (4H, d, J=3.2 Hz), 4.66 (1H, t, J=8.4 Hz), 5.71 (1H, s). ESI MS: 488.4 ([M+H]⁺).

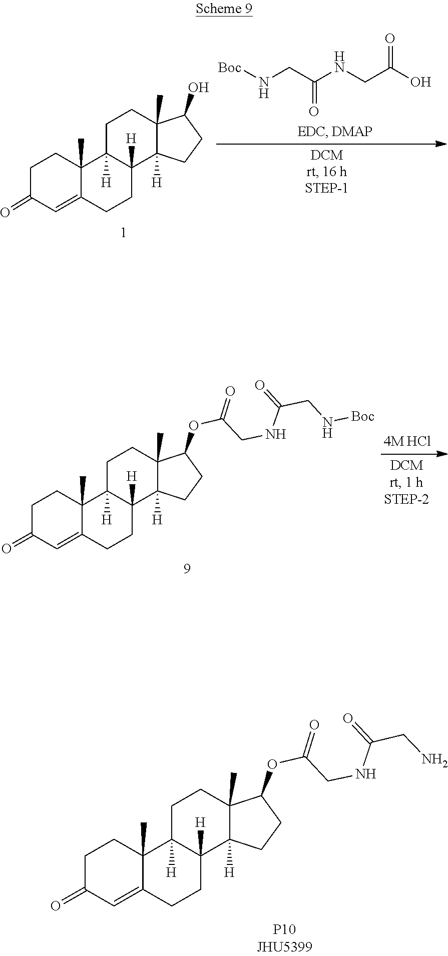

### Step 1:

To a solution of compound 1 (2.00 g, 6.93 mmol, 1.0 equiv.) and 2-[[2-(tert-butoxycarbonylamino)acetyl]amino]acetic acid (4.83 g, 20.8 mmol, 3.0 equiv.) in DCM (30 mL) were added EDC (6.65 g, 34. 7 mmol, 5.0 equiv.) and DMAP (424 mg, 3.47 mmol, 0.5 equiv). The mixture was stirred at 25° C. for 16 h. The mixture was concentrated under reduced pressure. The crude product was purified by reversed-phase HPLC [MeCN/H₂O=0_100% (0.1% HCl)]. Compound 9 was obtained as a brown solid (1.0 g) in 27% yield. ESI MS: 447.3 ([M+H]⁺).

### Step 2:

(8R,9S,10R,13S,14S,17S)-10,13-Dimethyl-3-oxo-2,3,6,7,8,9,10,11,12,13,14,15,16,17-tetradecahydro-1H-cyclopenta[α]phenanthren-17-yl glycylglycinate (P10, JHU5399)

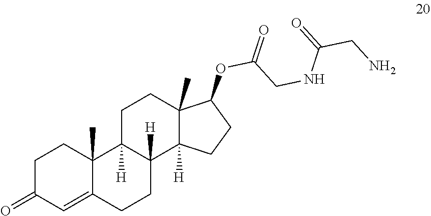

Compound 9 (500 mg, 0.927 mmol, 1.0 equiv) was dissolved in 4M HCl in dioxane (232 μL, 1.0 equiv.) and the mixture was stirred at 25° C. for 1 h. The mixture was concentrated under reduced pressure. The residue was purified by prep-HPLC (column: Welch Xtimate C18 150×25 mm×5 um; mobile phase: [water (HCl)-MeCN]; B %: 13%-43%, 9 min). Compound P10 (JHU5399) was obtained as a colorless solid (221 mg) in 54% yield as a salt of hydrochloric acid.

¹H-NMR (400 MHz, d₄-MeOD): 0.90 (3H, s), 1.07 (2H, d, J=3.6 Hz), 1.09-1.16 (1H, m), 1.16-1.23 (1H, m), 1.24 (3H, s), 1.39-1.53 (2H, m), 1.56-1.65 (2H, m), 1.66-1.75 (3H, m), 1.82 (1H, td, J=3.2, 12.8 Hz), 1.88-1.94 (1H, m), 2.05-2.13 (1H, m), 2.14-2.22 (1H, m), 2.25-2.35 (2H, m), 2.44-2.54 (2H, m), 3.74 (2H, s), 4.03 (2H, d, J=2.4 Hz), 4.68 (1H, t, J=8.4 Hz), 5.72 (1H, s). ESI MS: 403.1 ([M+H]⁺).

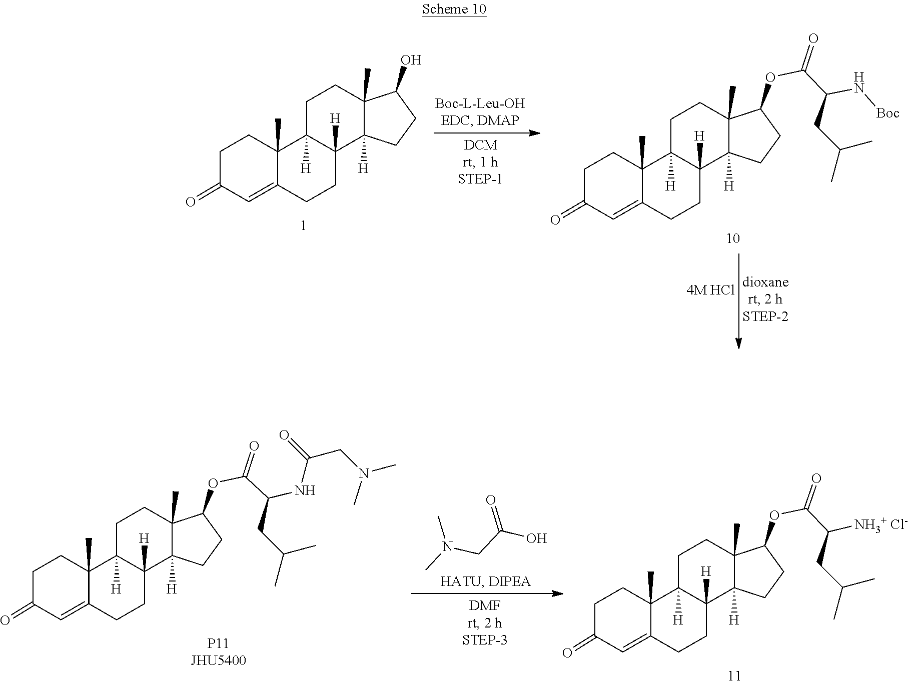

### Step 1:

To a mixture of testosterone 1 (500 mg, 1.73 mmol, 1.0 equiv), Boc-L-Leu-OH (800 mg, 3.46 mmol, 2.0 equiv.) and EDC (995 mg, 5.19 mmol, 3.0 equiv) in DCM (10 mL) was added DMAP (106 mg, 0.865 mmol, 0.5 equiv.). The mixture was stirred at 25° C. for 1 h. The reaction mixture was diluted with H₂O (20 mL) and extracted with DCM (3×20 mL). The combined organic layers were dried over anhydrous Na₂SO₄, filtered and concentrated under reduced pressure. The residue was purified by flash silica gel chromatography (ISCO®; 20 g SepaFlash® Silica Flash Column, Eluent of 0˜25% EtOAc/Petroleum ether gradient @ 60 mL/min). Compound 10 was obtained as a colorless solid (700 mg) in 81% yield. ESI MS: 502.3 ([M+H]⁺).

### Step 2:

Compound 10 (700 mg, 1.40 mmol, 1 equiv.) was dissolved in 4M HCl in dioxane (9.80 mL, 28 equiv.) and the mixture was stirred at 25° C. for 2 h. The reaction mixture was concentrated under reduced pressure to give a residue. The crude product 11 was obtained as a colorless solid (600 mg) in 96% yield as a salt of hydrochloric acid and was used to the following step without purification. ESI MS: 402.2 ([M+H]⁺).

### Step 3:

(8R,9S,10R,13S,14S,17S)-10,13-Dimethyl-3-oxo-2,3,6,7,8,9,10,11,12,13,14,15,16,17-tetradecahydro-1H-cyclopenta[α]phenanthren-17-yl dimethylglycyl-L-leucinate (P11,

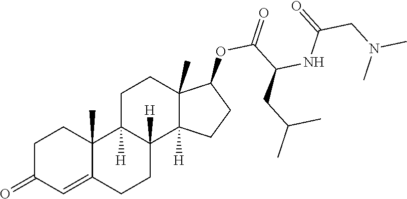

To a mixture of compound 11 (600 mg, 1.37 mmol, 1.0 equiv.) and 2-(dimethylamino) acetic acid (283 mg, 2.74 mmol, 2.0 equiv.) in DMF (6 mL) were added HATU (781 mg, 2.05 mmol, 1.5 equiv.) and DIPEA (531 mg, 716 μL, 4.11 mmol, 3.0 equiv) and the mixture was stirred at 25° C. for 2 h. The reaction mixture was filtered and concentrated under reduced pressure to give a residue. The residue was purified by prep-HPLC (column: Welch Xtimate C18 150×25 mm×5 um; mobile phase: [water (HCl)-MeCN]; B %: 25%-55%, 9 min). Compound P11 (JHU5400) was obtained as a yellow solid (255 mg) in 35% yield.

¹H-NMR (400 MHZ, d₄-MeOD): 0.85-0.97 (6H, m), 0.98-1.10 (5H, m), 1.11-1.18 (1H, m), 1.18-1.32 (4H, m), 1.35-1.52 (2H, m), 1.55-1.83 (9H, m), 1.87-1.95 (1H, m), 2.04-2.12 (1H, m), 2.13-2.23 (1H, m), 2.25-2.36 (2H, m), 2.43-2.56 (2H, m), 2.94 (6H, s), 4.00 (2H, q, J=15.6 Hz), 4.41-4.53 (1H, m), 4.59-4.71 (1H, m), 5.72 (1H, s), 8.76 (1H, d, J=7.6 Hz). ESI MS: 487.3 ([M+H]⁺).

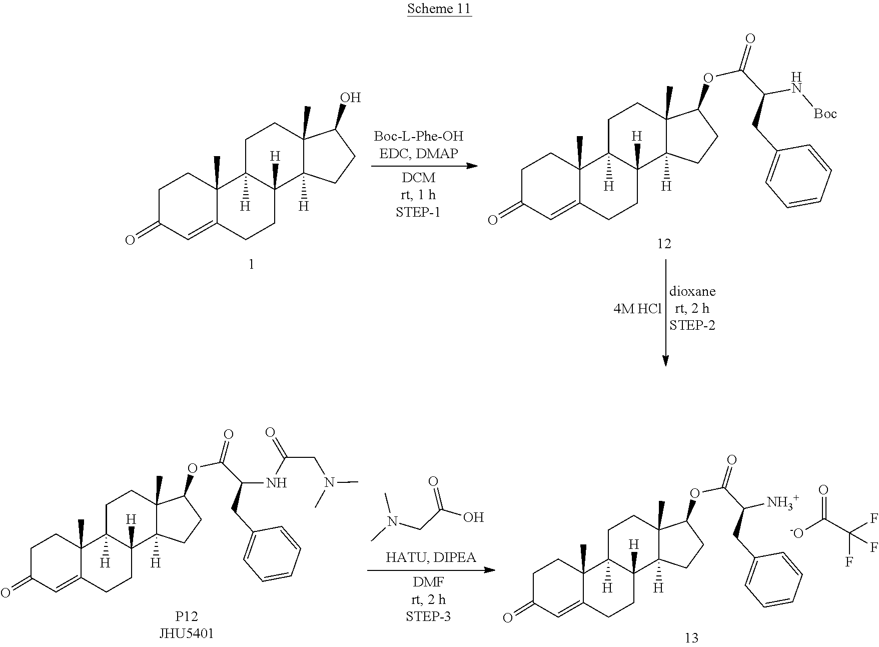

### Step 1:

To a mixture of testosterone 1 (500 mg, 1.73 mmol, 1.0 equiv.), Boc-L-Phe-OH (920 mg, 3.47 mmol, 2.0 equiv.) and EDC (997 mg, 5.20 mmol, 3.0 equiv.) in DCM (10 mL) was added DMAP (106 mg, 0.867 mmol, 0.5 equiv.) and the mixture was stirred at 25° C. for 2 h. The reaction mixture was diluted with H₂O (20 mL) and extracted with DCM (3×20 mL). The combined organic layer was dried over anhydrous Na₂SO₄, filtered and concentrated under reduced pressure. The residue was purified by flash silica gel chromatography (ISCO®; 20 g SepaFlash® Silica Flash Column, eluent of 0˜25% EtOAc/Petroleum ether gradient @ 60 mL/min). Compound 12 was obtained as a yellow solid (700 mg) in 75% yield. ESI MS: 536.2 ([M+H]⁺).

### Step 2:

Compound 12 (700 mg, 1.31 mmol, 1 equiv) was dissolved in DCM (6 mL) and TFA (700 μL, 9.45 mmol, 7.2 equiv.) was added. The mixture was stirred at 25° C. for 1 h. The reaction mixture was concentrated under reduced pressure. Compound 12 was obtained as a yellow oil (700 mg) in 97% yield as a TFA salt and was used to next step without further purification. ESI MS: 436.3 ([M+H]⁺).

### Step 3:

(8R,9S,10R,13S,14S,17S)-10,13-Dimethyl-3-oxo-2,3,6,7,8,9,10,11,12,13,14,15,16,17-tetradecahydro-1H-cyclopenta[α]phenanthren-17-yl dimethylglycyl-L-phenylalaninate (P12,

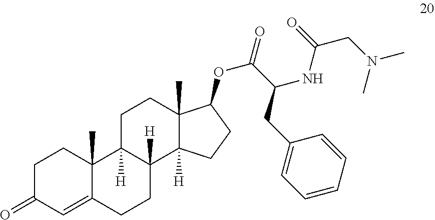

To a solution of compound 12 (500 mg, 0.910 mmol, 1.0 equiv.) in DMF (2 mL) were added 2-(dimethylamino) acetic acid (188 mg, 1.82 mmol, 2.0 equiv.), HATU (519 mg, 1.36 mmol, 1.5 equiv.) and DIPEA (353 mg, 475 μL, 2.73 mmol, 3.0 equiv.). The mixture was stirred at 25° C. for 2 h. The reaction mixture was filtered and the filtrate was concentrated under reduced pressure. The residue was purified by prep-HPLC (column: Phenomenex Luna C18 200×40 mm×10 um; mobile phase: [water (HCl)-ACN]; B %: 30%-60%, 9 min). Compound P12 (JHU5401) was obtained as a colorless solid (220 mg) in 41% yield.

¹H-NMR (400 MHZ, CDCl₃): 0.83 (3H, s), 0.91-1.13 (4H, m), 1.16 (1H, d, J=2.0 Hz), 1.21 (3H, s), 1.32-1.49 (3H, m), 1.53-1.65 (4H, m), 1.66-1.74 (3H, m), 1.86-1.92 (3H, m), 1.99-2.23 (2H, m), 2.26-2.52 (4H, m), 2.95-3.09 (1H, m), 3.16-3.31 (1H, m), 3.80-3.99 (2H, m), 4.61 (1H, t, J=8.4 Hz), 4.75-4.87 (1H, m), 5.76 (1H, s), 7.21-7.27 (4H, m), 7.30-7.35 (1H, m), 7.47 (1H, d, J=6.4 Hz), 8.50-9.05 (1H, m). ESI MS: 521.3 ([M+H]⁺).

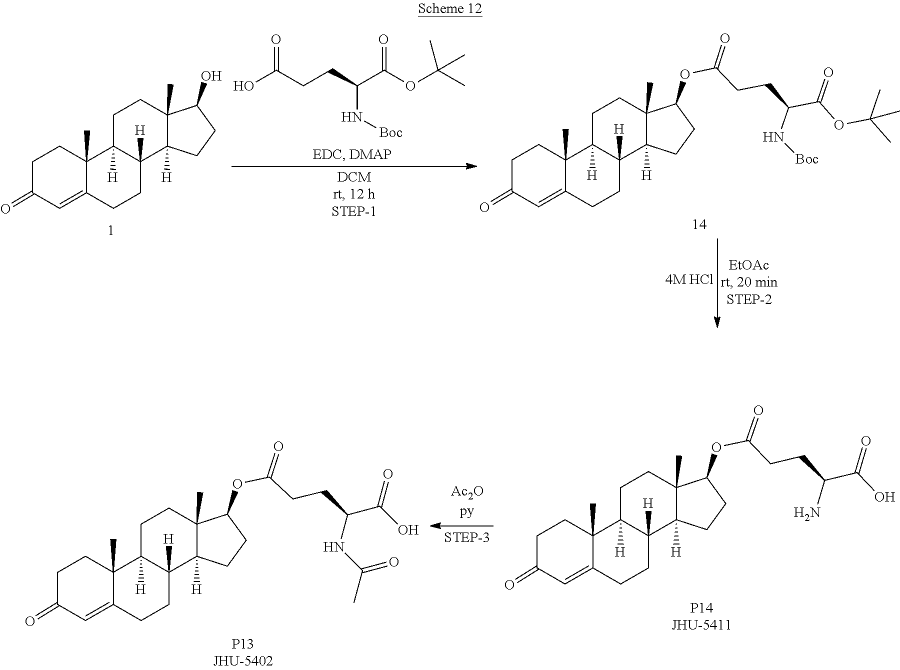

### Step 1:

To a solution of testosterone 1 (1.00 g, 3.47 mmol, 1.0 equiv.) in DCM (20 mL) were added (4S)-5-tert-butoxy-4-(tert-butoxycarbonylamino)-5-oxo-pentanoic acid (2.10 g, 6.93 mmol, 2.0 equiv.), EDC (1.99 g, 10.4 mmol, 3.0 equiv.) and DMAP (127 mg, 1.04 mmol, 0.3 equiv.). The mixture was stirred at 25° C. for 12 h. The reaction mixture was filtered and the filtrate was concentrated under reduced pressure. The residue was purified by prep-HPLC (water (0.1% NH₃·H₂O)/MeCN=1/0 to 0/1). Compound 14 was obtained as a colorless solid (2.5 g) in quantitative yield. ESI MS: 399.1 ([M+H]⁺).

### Step 2:

(S)-2-Amino-5-(((8R,9S,10R,13S,14S,17S)-10,13-dimethyl-3-oxo-2,3,6,7,8,9,10,11,12,13,14,15,16,17-tetradecahydro-1H-cyclopenta[α]phenanthren-17-yl)oxy)-5-oxopentanoic acid (P14, JHU5411)

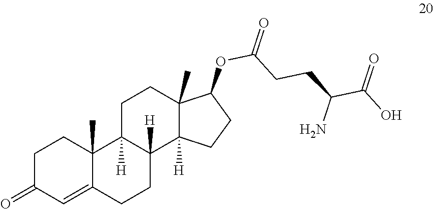

Compound 14 (2.50 g, 4.36 mmol, 1.0 equiv.) was dissolved in 4M HCl in EtOAc (15 mL, 13.8 equiv.) and the resulting mixture was stirred at 25° C. for 20 min. The reaction mixture was concentrated under reduced pressure. The crude product was triturated with EtOAc at 25° C. and filtered. The filter cake was dried in vacuum. Compound P14 (JHU5411) was obtained as a colorless solid (1.80 g) in 89% yield.

¹H-NMR (400 MHZ, d₆-DMSO): 0.79 (3H, s), 0.86-1.01 (2H, m), 1.02-1.20 (5H, m), 1.26-1.41 (2H, m), 1.42-1.72 (6H, m), 1.73-1.83 (1H, m), 1.92-2.28 (6H, m), 2.31-2.47 (3H, m), 2.53-2.61 (1H, m), 3.91 (1H, brs), 4.55 (1H, t, J=8.4 Hz), 5.63 (1H, s), 8.39 (2H, brs). ESI MS: 418.1 ([M+H]⁺).

### Step 3:

(S)-2-Acetamido-5-(((8R,9S,10R,13S,14S,17S)-10,13-dimethyl-3-oxo-2,3,6,7,8,9,10,11,12,13,14,15,16,17-tetradecahydro-1H-cyclopenta[α]phenanthren-17-yl)oxy)-5-oxopentanoic acid (P13, JHU5402)

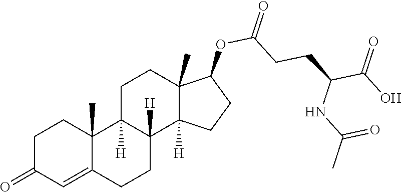

Compound P14 (700 mg, 1.68 mmol, 1.0 equiv.) was dissolved in pyridine (7 mL), Ac₂O (342 mg, 314 μL, 3.35 mmol, 2.0 equiv.) was added and the mixture was stirred at 25° C. for 16 h. The reaction mixture was adjusted to pH=3 with HCl (1.2 M) and then the mixture was diluted with H₂O (10 mL) and extracted with EtOAc (3×10 mL). The combined organic layers were concentrated under reduced pressure to give a residue. The residue was purified by prep-HPLC (column: Welch Xtimate C18 150×25 mm×5 um; mobile phase: [water (HCl)-MeCN]; B %: 26%-56%, 9 min), followed by prep-TLC (SiO₂, Petroleum ether: Ethyl acetate=0:1). Compound P13 (JHU5402) was obtained as a yellow solid (202 mg) in 24% yield.

¹H-NMR (400 MHZ, d₆-DMSO): 0.79 (3H, s), 0.87-1.01 (2H, m), 1.03-1.19 (5H, m), 1.27-1.40 (2H, m), 1.45-1.70 (6H, m), 1.74-1.83 (1H, m), 1.93-2.10 (4H, m), 2.12-2.28 (2H, m), 2.33-2.48 (3H, m), 2.53-2.62 (1H, m), 3.91 (1H, brs), 4.55 (1H, t, J=8.4 Hz), 5.63 (1H, s), 8.39 (2H, brs). ESI MS: 460.2 ([M+H]⁺).

### Example 2

### Biological Methods

### 2.1 In Vitro Metabolic Stability Assessment

In vitro metabolic stability assessment was performed using brain homogenates. For the brain homogenates washed tissues were diluted 10-fold in 0.1-M potassium phosphate buffer and homogenized using a probe sonicator. To evaluate the stability of the intact prodrug over time, the tissue matrix was aliquoted (1 mL) and then spiked with 10 μM of corresponding prodrugs (P1, P3, P5, P6, P7, P8, P9, P10, P11, P12, P13 and P14) followed by incubation in an orbital shaker at 37° C. for 1h (in triplicate). Samples from each incubation at predetermined time points (0, 0.5, and 1h) were quenched with 3X volumes of acetonitrile containing the internal standard (IS; losartan: 0.5 μm). Samples were vortex-mixed for 30 secs and centrifuged at 10,000 RPM for 10 min at 4° C. Disappearance of the intact compound and release of Testosterone were measured using liquid chromatography tandem mass spectrometry (LC-MS; full scan mode).

### 2.2 In Vivo Pharmacokinetic Studies in Female Rats

Female wistar rats (n=3) were used to study the pharmacokinetic profiles of prodrugs (P1, P3, P4, P5, P6, P7, P8, P9, P10, P11, P12, P13 and P14) and testosterone. All prodrugs were administered intranasally (IN) at a dose of 1.1 mg/kg equivalent of Testosterone using a formulation of 5% Tween-80, 5% DMSO and 90% PBS containing 10 mM HEPES. Blood and brain were collected after sacrificing the animals at predetermined time point. Plasma was harvested from blood by centrifugation at 3,000 RPM for 15 min and stored at −80° C. Brain tissues were harvested following blood collection and immediately snap frozen in liquid nitrogen and stored at −80° C. until LC-MS/MS analysis. Calibration standards were prepared using naïve female rat plasma or brain with additions of respective prodrugs. For quantifying the released testosterone in the pharmacokinetic samples, plasma samples (20 μL) were processed by protein precipitation method by addition of 200 μL of acetonitrile containing internal standard (losartan: 0.5 μM), followed by vortex-mixing for 30 s and then centrifugation at 10,000 RPM for 10 min at 4° C. Brain tissues were diluted 1:5 w/v with acetonitrile containing losartan (0.5 μm) and homogenized, followed by vortex-mixing and centrifugation at 10,000 RPM for 10 min at 4° C. A 50 μL aliquot of the supernatant was diluted with 50 μL of water and transferred to 250 μL polypropylene autosampler vials sealed with teflon caps. Then, 2 μL of the sample was injected into the LC-MS/MS system for analysis.

### Example 3

### Results

### 3.1 Prodrugs Stability in Rat Brain.

The metabolic stability of prodrugs (n=3) was evaluated using rat brain homogenates. The rat brain was homogenized using 100-mM phosphate buffer with 1:10 dilution (w/v). 10 μM concentration of the compounds was incubated at 37° C. and stability was evaluated from 0 hour to 1 hour. After incubation samples were precipitated using acetonitrile containing internal standard and quantified using high-resolution LC-MS/MS.

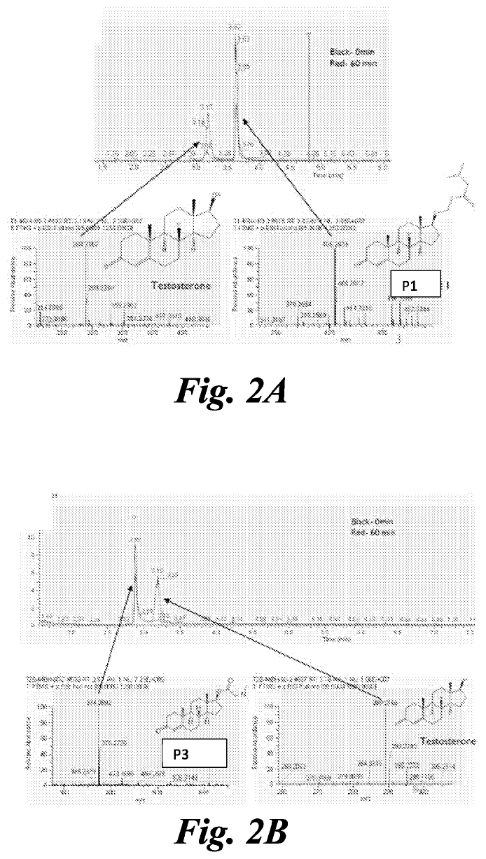

Prodrugs (P5, P6, P8, P9, P11, P12, P13 and P14) were found to be stable in rat brain homogenate, while P3, P7, and P10 were moderately stable and P1 is 20% stable in brain homogenate (FIG. 2)

### 3.2 Prodrugs Showed Improved Brain Penetration Compared to Testosterone Following Intranasal (IN) Administration

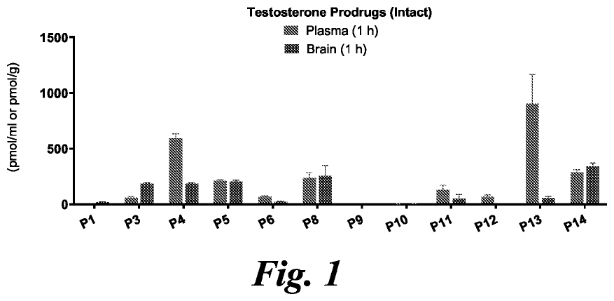

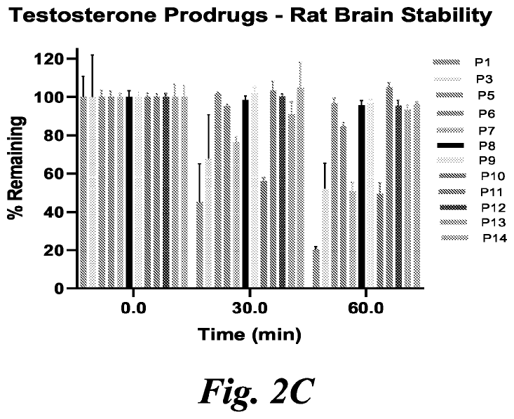

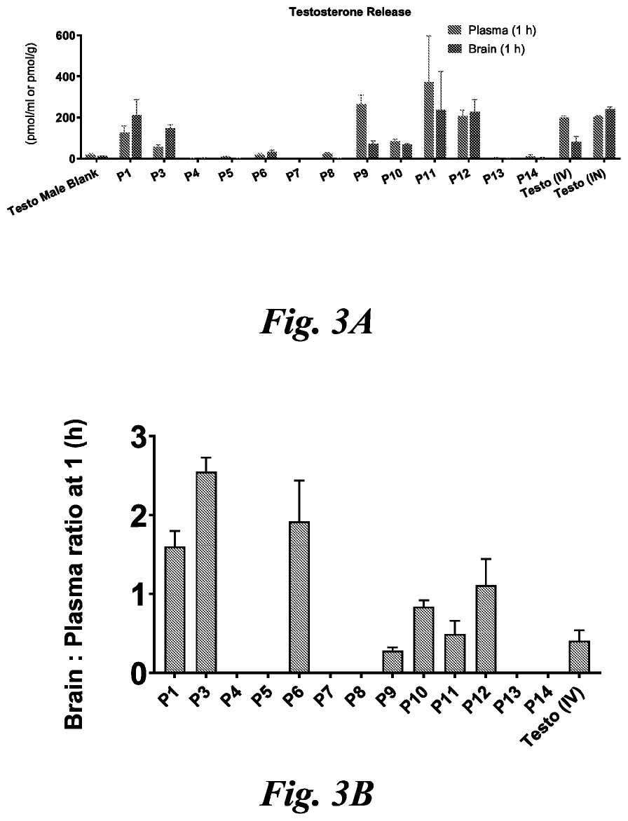

The in vivo pharmacokinetics of prodrugs (P1, P3, P4, P5, P6, P7, P8, P9, P10, P11, P12, P13 and P14) were evaluated in female rats. Rats were dosed (1.1 mg/kg equivalent of Testosterone), and plasma and brain levels of released testosterone were measured at 1h post dose. The results from the pharmacokinetic analysis are presented in FIG. 3A and FIG. 3B. Prodrugs P1, P3, P11 and P12 showed good levels of testosterone release in brain, and the levels were comparable to testosterone administration (150-250 pmol/g). The plasma levels of prodrugs P1, P3, P12 were lower when compared with the testosterone (as hypothesized) following IN administration. Prodrug P4, P5, P8, P13 and P14 showed low levels of testosterone release attributed to its higher stability (FIG. 1). Prodrug P3 demonstrated a 2-fold increase (1.2 versus 2.5) in the brain-to-plasma ratio compared to Testosterone (FIG. 3B). Although the prodrug P6 showed low levels of Testosterone release, the brain-to-plasma ratio was 2-fold higher compared to Testosterone (1.2 versus 2).

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A compound of formula (I): 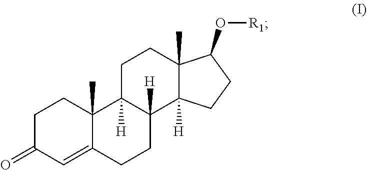
wherein:
R₁ is selected from —(CH₂)ₙ—O—C(═O)—O—R₂, —C(═O)—(CH₂)ₘ—NR₃R₄, —C(═O)—CH(R₅)—NR₆—C(═O)—CH(R₇)—R₈, —C(═O)—R₉, —C(═O)—(CH₂)ₘ—NR₁₀—C(═O)—(CH)ₚ—NR₁₁R₁₂, —C(═O)—(CH₂)ₘ—NR₁₃—(C═O)—(CH)_{q}—NR₁₄—C(═O)—(CH₂)ₚ—NR₁₅R₁₆, —C(═O)—CH(R₁₇)—NR₁₈—C(═O)—(CH₂)ₚ—NR₁₉R₂₀, —C(═O)—(CH₂)ₘ—CHR₂₁—C(═O)—OH;
each n, m, and p is independently an integer selected from 0, 1, 2, 3, 4, 5, 6, 7, and 8;
R₂, R₃, R₄, R₅, and R₅ are each independently straightchain or branched C₁-C₄ alkyl, or R₃ and R₄ together with the nitrogen atom to which they are bound form a substituted or unsubstituted 6-membered cycloheteroalkyl ring;
R₆, R₁₀, R₁₁, R₁₂, R₁₃, R₁₄, R₁₅, and R₁₆, R₁₈, and R₁₉, and R₂₀ are each independently H or C₁-C₄ alkyl;
R₇ is —NR₂₂R₂₃, wherein R₂₂ and R₂₃ are each independently H or C₁-C₄ alkyl;
R₉ is substituted or unsubstituted straightchain or branched C₁-C₄ alkyl;
R₁₇ is substituted or unsubstituted straightchain or branched C₁-C₈ alkyl;
R₂₁ is —NR₂₂R₂₃, wherein R₂₂ and R₂₃ are each independently H or —C(═O)—OR₂₄, wherein R₂₄ is C₁-C₄ alkyl; or
a pharmaceutically acceptable salt thereof.

2. The compound of claim 1, wherein R₁ is —(CH₂)ₙ—O—C(═O)—O—R₂, wherein n is 1 and R₂ is selected from methyl, ethyl, n-propyl, isopropyl, n-butyl, isobutyl, sec-butyl, and tert-butyl.

3. The compound of claim 1, wherein R₁ is —C(═O)—(CH₂)ₘ—NR₃R₄, wherein m is 1 and R₃ and R₄ are each independently selected from methyl, ethyl, n-propyl, isopropyl, n-butyl, isobutyl, sec-butyl, and tert-butyl, or R₃ and R₄ together with the nitrogen atom to which they are bound form a 4-methylpiperazinyl or morpholinyl ring.

4. The compound of claim 1, wherein R₁ is —C(═O)—CH(R₅)—NR₆—C(═O)—CH(R₇)—R₈, wherein R₆ is H, R₇ is —NH₂, and R₅ and R₈ are each independently selected from methyl, ethyl, n-propyl, isopropyl, n-butyl, isobutyl, sec-butyl, and tert-butyl.

5. The compound of claim 1, wherein R₁ is —C(═O)—R₉, wherein R₉ is C₁-C₄ alkyl substituted with halogen.

6. The compound of claim 1, wherein R₁ is —C(═O)—(CH₂)ₘ—NR₁₀—C(═O)—(CH)ₚ—NR₁₁R₁₂, wherein m and p are each 1, R₁₀ is H and R₁₁ and R₁₂ are each C₁-C₄ alkyl.

7. The compound of claim 1, wherein R₁ is —C(═O)—(CH₂)ₘ—NR₁₃—(C═O)—(CH)_{q}—NR₁₄—C(═O)—(CH₂)ₚ—NR₁₅R₁₆, wherein m, p, and q are each 1, R₁₃ and R₁₄ are each H and R₁₅ and R₁₆ are each C₁-C₄ alkyl.

8. The compound of claim 1, wherein R₁ is —C(═O)—CH(R₁₇)—NR₁₈—C(═O)—(CH₂)ₚ—NR₁₉R₂₀, wherein p is 1, R₁₈ is H, R₁₉ and R₂₀ are each C₁-C₄ alkyl, and R₁₇ is selected from methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, and benzyl.

9. The compound of claim 1, wherein R₁ is —C(═O)—(CH₂)ₘ—CHR₂₁—C(═O)—OH, wherein m is 2 and R₂₁ is —NH₂ or —NH(COOCH₃).

10. The compound of claim 1, wherein the compound of formula (I) is selected from:

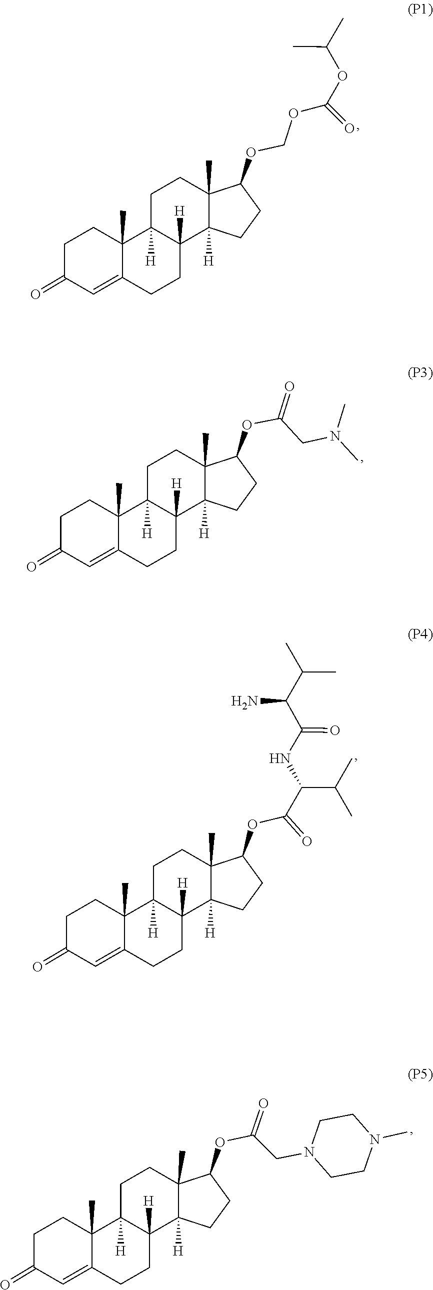

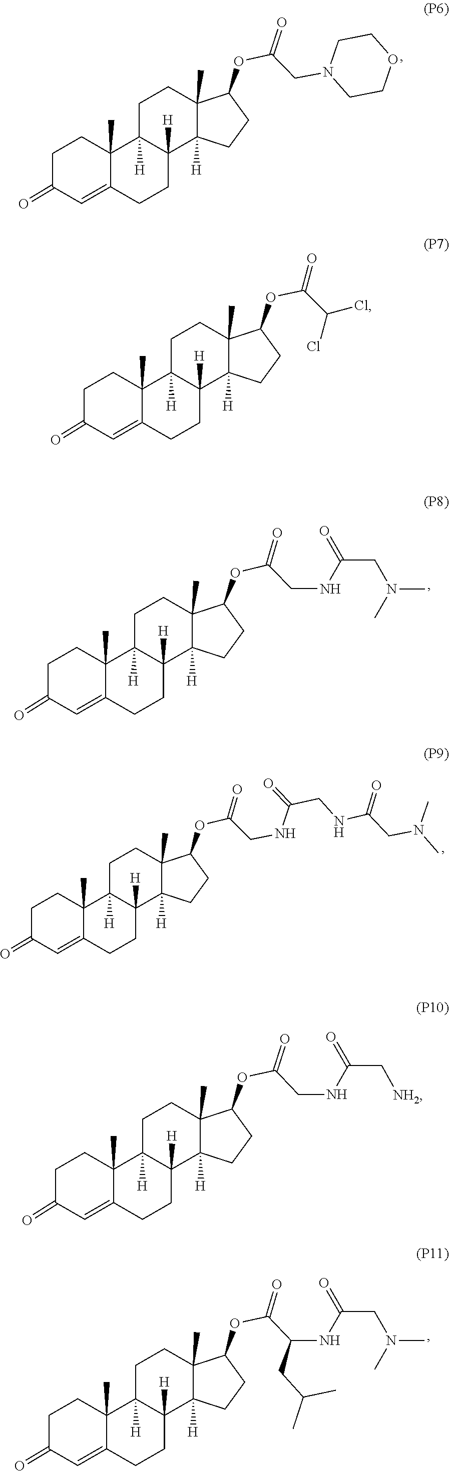

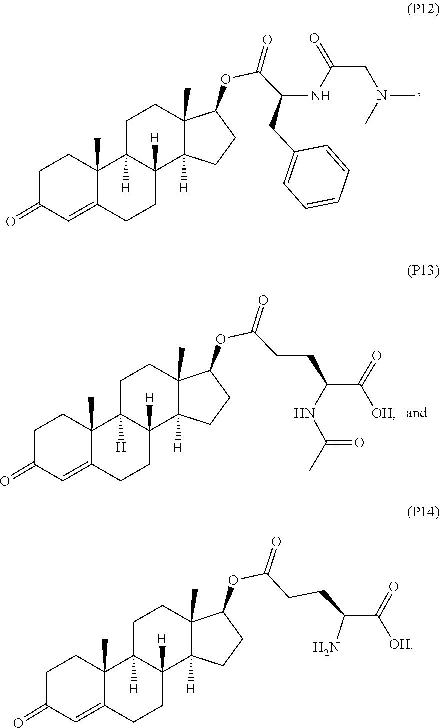

11. A pharmaceutical formulation comprising the compound of any one of claim 1 and a pharmaceutically acceptable carrier.

12. The pharmaceutical formulation of claim 11, wherein the formulation is formulated for intranasal delivery.

13. A method for treating a subject for prostate cancer, the method comprising administering to the subject a compound of formula (I) of any one of claims 1-10, or a pharmaceutical formulation of claim 11, in combination with one or more additional therapies for prostate cancer.

14. The method of claim 13, wherein the prostate cancer comprises stage IV prostate cancer or a metastatic prostate cancer.

15. The method of claim 13, wherein the one or more additional therapies for prostate cancer comprises androgen deprivation therapy (ADT).

16. The method of claim 15, wherein the ADT is achieved through orchiectomy or administration of a luteinizing hormone-releasing hormone agonist/antagonist.

17. The method of any one of claim 13, wherein the method alleviates, improves, or reduces a risk of developing one or more conditions associated with ADT selected from a bone fracture, a cardiovascular event, a cognitive deficit, a sexual dysfunction, and severe fatigue.

18. The method of claim 13, wherein the compound of formula (I) is administered intranasally.
